(19) United States
(12) Patent Application Publication
(10) Pub. No.: US 2003/0180948 A1
(43) Pub. Date: Sep. 25, 2003

(54) Superficial zone protein and methods of making and using same

(76) Inventors: Jeff T. Hutchins, Cumming, GA (US); Klaus E. Kuettner, Chicago, IL (US); Thomas M. Schmid, Downers Grove, IL (US); Barbara L. Schumacher, Cardiff by the Sea, CA (US); Jui-Lan Su, Durham, NC (US); Eric P. Dixon, Durham, NC (US)

Correspondence Address: Tina Williams McKeon, NEEDLE & ROSENBERG, P.C., The Candler Building, Suite 1200, 127 Peachtree Street, N.E., Atlanta, GA, 30303-1811, US

(21) Appl. No.: 10/038,694

(22) Filed: Dec. 31, 2001

Related U.S. Application Data
(60) Provisional application No. 60/258,920, filed on Dec. 29, 2000.

(51) Int. Cl. C12N 15/866; C12N 5/08

(52) U.S. Cl. 435/456; 435/366

(57) ABSTRACT

The present invention relates to SZP polypeptides and nucleic acids encoding the polypeptides. The invention further relates to methods of making and using the polypeptides and nucleic acids. Also, the invention relates to immortalized chondrocytes or SZP-producing cells and methods of using the cells. The invention also relates to a method of culturing immortalized or non-immortalized chondrocytes in serum-free medium. The invention also relates to a method of imaging an articular surface or synovium of a joint using detectably tagged SZP or detectably tagged SZP binding proteins.

This application claims priority to U.S. Provisional Patent No. 60/258,920, filed Dec. 29, 2000, which is incorporated herein by reference in its entirety.

This invention was made with government support under 2P50-AR39239 awarded by the National Institute for Arthritis and Musculoskeletal Diseases of the National Institutes of Health. The government may have certain rights in the invention.

### BACKGROUND OF THE INVENTION

1. Field of the Invention

This invention relates generally to superficial zone protein and its production and uses, including therapeutic uses in treatment and imaging in degenerative joint disease.

2. Background Art

The major function of articular cartilage at the end of long bones is to provide a frictionless surface for the efficient movement of articulating bones. Over the last thirty years, cartilage research has focused on the material properties of cartilage tissue, with little emphasis on molecules that lubricate the articular cartilage surface. To date, no cartilage molecule or combination of molecules has been identified that can account for the nearly frictionless properties of the articular cartilage surface.

Articular cartilage is a highly organized, heterogeneous, avascular, resilient, weight-bearing tissue that covers the ends of bones in diarthrodial (synovial) joints. The organizational arrangement of articular cartilage is marked by zonal differences. For example, the superficial zone of adult articular cartilage is distinctly different from the middle, deep, and calcified zones of the underlying cartilage in cellularity, morphology, matrix and macromolecular composition (which includes the presence of gene products made in different zones), macromolecular organization, and material properties. Many of the first changes to develop in degenerative joint disease like osteoarthritis (OA) take place at the articular surface within the superficial zone of articular cartilage (Guilak et al. (1994); Hollander et al. (1995); Hauselmann et al. (1996); Aigner et al. (1997); Freemont et al. (1997); Towle et al. (1997); Ohta et al. (1998); Panula et al. (1998)). Two major components of articular cartilage are aggrecan and type II collagen. Hollander et al. (1995) have reported that damage to type II collagen in osteoarthritis starts at the articular surface and extends deeper into the cartilage with progressive degeneration. Likewise, Guilak et al. (1994) have reported that a major event in early OA is the disruption of the collagen network in the superficial zone of articular cartilage. Lark et al. (1997) report that aggrecan degradation by aggrecanase and matrix metalloproteinases is most severe in the superficial zone of articular cartilage. Thus, damage to two main components of articular cartilage, collagen type II and aggrecan, starts at the surface and proceeds deeper into the cartilage in OA.

Another difference in the various cartilage zones is differential synthesis of a proteoglycan called superficial zone protein (SZP) or proteoglycan 4, which is synthesized and secreted by chondrocytes in the superficial zone of articular cartilage but is not synthesized or secreted by chondrocytes in the deeper zones of the tissue (Schumacher et al. (1994)). SZP, which is homologous to human megakaryocyte stimulating factor precursor (MSF) and camptodactyly-arthropathy-coxa vara-pericarditis (CACP) protein, has an apparent molecular weight of 345 kDa and is substituted with keratan sulfate and chondroitin sulfate glycosaminoglycan chains (Schumacher et al. (1994)). Removal of the glycosaminoglycan side chains results in minimal change in molecular weight, which suggests that SZP has only small glycosaminoglycan chains on its core protein and that it is not an aggrecan metabolite (Schumacher et al. (1999)). SZP contains large (76-78 repeats) and small (6-8 repeats) mucin-like O-linked oligosaccharide-rich repeat domains flanked by cysteine-rich N- and C-terminal domains (Flannery et al. (1999)). The protein core contains potential sites for N-linked oligosaccharide and glycosaminoglycan attachment, and a putative heparin-binding domain (Id.). The heparin binding domain is encoded by exon 4 of MSF (Merberg et al. (1993)). Chondrocytes in the superficial zone and cells of the synovial lining, in vivo and in vitro, have been shown to synthesize SZP (Schumacher et al. (1999)). Unlike other proteoglycan molecules, such as aggrecan, decorin, biglycan, and fibromodulin, very little SZP is retained in the matrix surrounding the chondrocytes (Schumacher et al. (1994)). The SZP proteoglycan present in synovial fluid has a lower molecular weight than SZP in the cartilage matrix, suggesting that it may be a different gene product, a splice variant, or a different glycosylated form of SZP produced by synovial cells as compared to SZP produced by chondrocytes or a proteoglycan which is partially degraded in the synovial fluid (Schumacher et al. (1999)).

SZP forms a thin layer on the surface of adult bovine articular cartilage but not fetal articular cartilage (Schumacher et al. (1999)). The thickness of the stained layer increases gradually near the junction of articular cartilage with synovium and the synovium also contains SZP (Schmid et al. (1994)). This accumulation on adult articular cartilage has been hypothesized to be due to entrapment of SZP in an acellular collagenous layer at the surface of articular cartilage (Schumacher et al. (1999)). The biosynthesis of SZP by chondrocytes has been shown to be upregulated by certain growth factors and cytokines, such as TGFβ and IGF-1, but down regulated by others, such as IL-1 (Flannery et al. (1999)). Furthermore, molecular defects in human SZP have been identified in individuals with camptodactyl-arthropathy-coxa vara-pericarditis syndrome (CACP), a very rare condition that can be marked by a proliferation of synovial cells, severe limitations in joint range of motion, and non-inflammatory pericarditis (Marcelino et al. (1999)). The function of various molecules like SZP in normal joints and pathologic joints, however, has not been elucidated.

Prior to the present invention, studying SZP and its function had proven difficult because it had to be purified from cartilage or primary cultures of chondrocytes. No renewable source of SZP had been identified and neither the full length amino acid sequence nor the nucleic acid sequence encoding SZP had been elucidated or expressed recombinantly.

### SUMMARY OF THE INVENTION

In accordance with the purposes of this invention, as embodied and broadly described herein, this invention, in one aspect, relates to the amino acid sequence of SZP and nucleic acids encoding SZP. The invention also relates to a method of promoting lubrication between two juxtaposed biological surfaces, or a biological surface and a biologically compatible surface, comprising contacting the surfaces with SZP, or fragments or derivatives of SZP, having lubricating properties, under conditions which allow the SZP, or the fragments or derivatives of SZP, to bind to the surfaces. More specifically, the present invention provides a method of promoting lubrication of an articular surface of a joint, comprising contacting the articular surface of the joint with SZP, or fragments or derivatives of SZP, having lubricating properties, under conditions that allow the SZP, or the fragments or derivatives of SZP, to bind to the articular surface.

In another aspect, the invention relates to immortalized SZP-producing cells and to immortalized chondrocytes derived from chondrocytes isolated from a specific zone of articular cartilage.

In yet another aspect, the invention relates to a method of repairing a cartilage defect comprising transplanting the immortalized chondrocytes into the region of the defect, under conditions that allow the chondrocytes to produce cartilage matrix.

In another aspect the invention relates to a method of culturing immortalized or non-immortalized chondrocytes in serum-free medium, comprising culturing the chondrocytes in the serum-free medium supplemented with insulin, transferrin, and selenium.

In yet another aspect the invention relates to methods of producing isolated SZP. In one embodiment, the method comprises the steps of culturing immortalized or non-immortalized chondrocytes in serum-free medium under conditions that allow expression and secretion of SZP; harvesting the medium from the cultured chondrocytes; and isolating SZP from the medium. In another embodiment, the method comprises recombinant expression.

The invention also relates to a method of imaging an articular surface or synovium of a joint, comprising contacting the articular surface or synovium of the joint with detectably tagged SZP, under conditions in which the detectably tagged SZP binds to the articular surface or synovium, and visualizing the detectable tag in a plurality of locations on the articular surface or synovium.

Additional advantages of the invention will be set forth in part in the description, which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawing, which is incorporated in and constitutes a part of this specification, illustrates an aspect of the invention and, together with the description, serves to explain the principles of the invention.

FIG. 1 shows the relative amount of cartilage lubrication by various agents, including phosphate buffered saline (PBS), bovine serum (BCS), synovial fluid (Synf) and dilutions of hyaluronic acid (HA) and SZP. Two samples of each agent were tested. Frictional force measurements were recorded while a glass plate was rotating against the articular surface of the distal phalageal bone of the middle toe of an adult rabbit at 180, 160, 140, 120, 100, 80, 60, 40, and 20 rpm for two minutes each. PBS and hyaluronic acid had minimal lubricating activity (i.e., the greatest frictional force) compared to the other agents, whereas undiluted synovial fluid had the highest lubricating activity. Undiluted serum and purified preparations of SZP also displayed lubricating activity.

FIG. 2 shows the results of an SZP sandwich ELISA, using lectin-6.79 mAb, with a SZP standard and three samples of synovial fluids, which are designated samples 115, 120, and 124. The X axis shows the concentration of SZP. The synovial fluids were diluted by two-fold serial dilutions starting with a 1:125 dilution.

FIG. 3 shows range of concentration of SZP in 50 synovial fluid samples assayed using the SZP sandwich ELISA. The results show the combined data for patients with degenerative joint disease and organ donors and the data for patients and donors separately.

FIG. 4 shows the effect of SZP antibody on lubrication by human synovial fluid. SZP antibody, either conjugated to Sepharose beads or in soluble form, reduced the lubricating activity of human synovial fluid (hsf). Human synovial fluid with conjugated antibody (the line with asterisks) shows higher frictional forces as compared to the negative PBS control (open circles, solid line). When 250 μg of soluble SZP mAb is added to the synovial fluid to make it 10%, the soluble antibody diminished the lubrication activity of the fluid (see open diamonds), even though an equal concentration of antibody did not affect the lubrication activity of the PBS control (open circle, dashed line).

FIG. 5 shows the ability of SZP to bind with macromolecules such as bovine serum albumin (BSA, Sigma) (solid circle), hyaluronan (HA, Sigma) (Open circle), fibronectin (FN, gift from Dr. Gene Homandberg) (solid triangle), pepsinized bovine collagen II (COL2) (solid square), and aggrecan-link protein-hyaluronan complex (RCS-A1 fraction) (PG-A1) (open diamond) isolated from the rat chondrosarcoma tumor and purified on an associative CsCl₂ gradient. The bound SZP was detected with an SZP monoclonal antibody, followed by a goat-anti-mouse IgG-HRP conjugate. After washing, the HRP activity was detected as an increase in color with an o-phenylenediamine substrate. SZP bound in a concentration-dependent and saturable manner to bovine serum albumin, hyaluronan, and fibronectin. There was diminished but detectable binding to the aggrecan-link protein-HA complex. The SZP did not bind to pepsinized collagen type II.

FIG. 6 shows the effect of SZP and other macromolecules on chondrocyte attachment. Bovine chondrocytes in a single-cell suspension were added to tissue culture plates coated overnight with 10 μg/ml of SZP, IgG, HA, gelatin, or no coating in a pH 9.5 coating buffer. Following incubation, the plates were washed in PBS, and fixed in 10% formalin, and the attached cells were counted. The SZP-coated plate had the smallest number of cells compared to other coated or non-coated (blank) plates.

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific methods or specific immortalized chondrocytes, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “an immortalized cell line” includes mixtures of cell lines, reference to “culturing chondrocytes” includes culturing one or more such chondrocytes, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally,” as used throughout, means that the subsequently described event or circumstance may or may not occur, and that the description includes instances where said event or circumstance occurs and instances where it does not.

As used throughout, by “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

The invention provides purified SZP and nucleic acids encoding SZP. As used throughout, “superficial zone protein” or “SZP” can include the full-length proteoglycan, the full length protein core, and variants of SZP (e.g., alternatively spliced variants). The invention further provides fusion proteins comprising SZP, and fragments or derivatives of SZP that maintain one or more functions of SZP. “SZP,” as used throughout, can include forms of SZP found in either plasma, serum or synovial fluid. Thus, SZP and its fragments or derivatives include glycosylated or non-glycosylated forms and non-reduced or reduced SZP, unless otherwise made clear by specific reference.

In one embodiment, the invention provides a purified SZP polypeptide comprising the amino acid sequences SEQ ID NO: 2 or SEQ ID NO: 3 or both, preferably with one or more mucin repeats between SEQ ID NO: 2 and SEQ ID NO: 3. By “one or more mucin repeats” is meant 1-200, or any amount in between. Each mucin repeat preferably comprises the amino acid sequence of SEQ ID NO: 11.

The invention also provides the amino acid sequences of the SZP polypeptide with one or more conservative amino acid substitutions, as shown in Table I. The invention further provides amino acid sequences having at least 80% identity to SEQ ID NO: 2, SEQ ID NO: 3, or a combination thereof.

“Fragments or derivatives of SZP, having lubricating properties,” preferably include one or more mucin-like domains (SZP exon 6). Such fragments or derivatives preferably include the carboxyl terminal of the SZP protein core. Such fragments or derivatives can include one or more somatomedin B domains (SZP exons 2 and 3), hemopexin domains (SZP exons 8 and 9), heparin-binding motifs. It is understood that “fragments or derivatives of SZP” include functional variants. These variants are produced by making amino acid substitutions, deletions, and insertions, as well as post-translational modifications. Variations in post-translational modifications can include variations in the type or amount of carbohydrate moieties on the SZP protein core or any fragment or derivative thereof. Variations in amino acid sequence may arise naturally as allelic variations (e.g., due to genetic polymorphism) or may be produced by human intervention (e.g., by mutagenesis of cloned DNA sequences), such as induced point, deletion, insertion and substitution mutants. These modifications can result in changes in the amino acid sequence, provide silent mutations, modify a restriction site, or provide other specific mutations.

Amino acid sequence modifications fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about 2 to 6 residues are deleted at any one site within the protein molecule. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues but may include multiple substitutions at different positions; insertions usually will be on the order of about from 1 to 10 amino acid residues but can be more; and deletions will range about from 1 to 30 residues, but can be more. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with Table 1 and are referred to as conservative substitutions.

TABLE 1
Amino Acid Substitutions

| Original Residue | Exemplary Substitutions |
|---|---|
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Iie | leu; val |
| Leu | ile; val |
| Lys | arg; gln |
| Met | leu; ile |
| Phe | met; leu; tyr |
| Ser | Thr |
| Thr | Ser |
| Trp | Tyr |
| Tyr | trp; phe |
| Val | ile; leu |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions that in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (Creighton, 1983), acetylation of the N-terminal amine and, in some instances, amidation of the C-terminal carboxyl. Modifications in SZP can also include modifications in glycosylation, including the presence, absence or modification of glycosaminoglycan (GAG) side chains.

In all mutational events, it is understood that the controlling aspect of the mutation is the function that the subsequent protein core or proteoglycan possesses. The preferred mutations are those that do not detectably change the lubricating function or that increase the lubricating function.

The invention provides isolated nucleic acids that comprise a nucleotide sequence that encodes the SZP polypeptide. In preferred embodiments, the nucleotide sequence comprises SEQ ID NO: 5, SEQ ID NO: 6, or both. In a preferred embodiment, the nucleotide sequence further comprise a sequence encoding one or more mucin domains, preferably between SEQ ID NO: 5 and SEQ ID NO: 6. Degenerate variants of the nucleic acids are also provided.

The invention also provides an expression vector comprising the nucleic acids operably linked to an expression control sequence and cultured cells comprising the vectors.

Also provided is an isolated nucleic acid comprising a sequence that hybridizes under highly stringent conditions to a hybridization probe having the nucleotide sequence of SEQ ID NO: 5 or its complement, SEQ ID NO: 6 or its complement, or both.

The hybridizing portion of the hybridizing nucleic acid is typically at least 15 (e.g., 20, 25, 30, 50, or more) nucleotides in length. The hybridizing portion of the hybridizing nucleic acid is at least 80% (e.g., 85, 90, 95, or 98%) identical to the sequence of a portion or all of a nucleic acid encoding SZP, or its complement. Hybridizing nucleic acids can be used, for example, as a cloning probe, a primer (e.g., a PCR primer), or a diagnostic or imaging probe. Nucleic acid duplex or hybrid stability is expressed as the melting temperature or Tm, which is the temperature at which a probe dissociates from a target DNA. This melting temperature is used to define the required stringency conditions. If sequences are to be identified that are related or substantially similar to the probe rather than identical to the probe, then it is useful to first establish the lowest temperature at which only homologous hybridization occurs with a particular concentration of salt (e.g., SSC or SSPE). Then assuming that 1% mismatching results in a 1° C. decrease in the Tm, the temperature of the final wash in the hybridization reaction is reduced accordingly. For example, if sequences having >95% identity with the probe are sought, the final wash temperature is decreased by 5° C. In practice, the change in Tm can be between 0.5° C. and 1.5° C. per 1% mismatch. Highly stringent conditions involve hybridizing at 68° C. in 5×SSC/5× Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and the target nucleic acid. Additional guidance regarding conditions is readily available in the art, for example, in Sambrook et al., 1989, Molecular Cloning, A Laboratory Manual, Cold Spring Harbor Press, N.Y. or in Ausubel et al. (eds), 1995, Current Protocols in Molecular Biology, (John Wiley & Sons, N.Y.) at Unit 2.10.

It is understood that one way to define any known variants and derivatives of the disclosed nucleic acids and polypeptides herein is through defining the variants and derivatives in terms of homology to specific known sequences. For example, SEQ ID NOS: 2 and 3 set forth particular sequences of an SZP polypeptide and SEQ ID NOS: 5 and 6 set forth particular nucleic acids that encode SZP. Specifically disclosed are variants of these and other nucleic acids and polypeptides herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology or percent identity to the stated sequence. Those of skill in the art readily understand how to determine the homology of two polypeptides or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by reference for at least material related to nucleic acid alignment.

The invention also provides a variety of uses for SZP and its fragments and derivatives. In one embodiment, the invention provides a method of inhibiting cell adhesion to a biological surface or a biologically compatible surface comprising contacting the biological surface with SZP, or a fragment or derivative of SZP having cell adhesion inhibiting properties, under conditions which allow SZP, or the fragment or derivative thereof, to inhibit cell adhesion to the surface. In a specific embodiment, the surface is an articular surface of a joint, and cell adhesion to the articular surface is inhibited.

The invention provides a method of modulating lubrication of an articular surface of a joint, comprising contacting the articular surface of the joint with an SZP binding protein, under conditions that allow SZP to bind to the SZP binding protein. In preferred embodiments, the SZP binding protein is selected from the group consisting of an SZP antibody, lectins, hyaluronan, fibronectin, and albumin. By “modulating lubrication” is meant either increasing or decreasing lubrication. In one embodiment, the interaction of the binding protein and SZP or its fragments or derivatives serves to increase lubrication. Such an increase in lubrication may be desired to reduce or prevent frictional forces and degenerative changes associated with such friction forces. A decrease in lubrication may be preferred when the surface of a prosthetic joint, for example, renders a joint unstable and increased friction forces are desired. Also provided is a method of modulating lubrication of a biological surface or a biologically compatible surface, comprising contacting the surface with an SZP binding protein, under conditions which allow SZP to bind to the SZP binding protein.

As used throughout, a “biological surface” is any naturally-occurring surface including, for example, epithelial tissue, connective tissue, muscle, neural tissue, cartilage, tendon, ligament, pericardium, and blood vessel. “Biologically compatible surfaces” include, for example, various biologically inert polymers and metals.

In one embodiment the invention provides a method of promoting lubrication between two juxtaposed surfaces (including, e.g., pericardial surfaces, articular joint surfaces, the surfaces of a tendon and bone, and the surfaces of a tendon and tendon sheath), comprising contacting the surfaces with SZP, or fragments or derivatives of SZP, having lubricating properties, under conditions which allow the SZP, or the fragments or derivatives of SZP, to bind to the surfaces. More specifically, the present invention provides a method of promoting lubrication of an articular surface of a joint, comprising contacting the articular surface of the joint with SZP, or fragments or derivatives of SZP, having lubricating properties, under conditions that allow the SZP, or the fragments or derivatives of SZP, to bind to the articular surface. Diminished lubrication capacity at the articular surface would be expected to cause greater surface wear of the cartilage, lesions and ultimately the failure of the joint. Thus, the present method aims to promote lubrication and reduce surface wear and degeneration of the joint. In a preferred embodiment, the method reduces the frictional force of the articular surface more than serum and hyaluronic acid reduce the frictional force of the articular surface.

In one embodiment, the method of promoting lubrication of an articular surface of a joint, comprising contacting the articular surface of the joint with SZP, or fragments or derivatives of SZP, further comprises contacting the articular surface of the joint with a protease inhibitor. Contacting with the protease inhibitor can be performed before, after, or during contacting with the SZP or fragment or derivative thereof. Optionally, the contacting step with the protease inhibitor can be repeated more than once. The purpose of the protease inhibitor is to promote and prolong the activity of the SZP or fragment or derivative thereof by reducing protein degradation. The protease inhibitor may act directly by reducing degradation of the SZP or fragment itself or by reducing degradation of an enzyme that enhances the activity of the SZP or fragment thereof. In an alternative embodiment of the method, other molecules are used to augment the effect of SZP. Such a molecule can include enzymes such as proteases or glycosidases.

By “lubrication” and “lubricating properties” is meant a reduction in friction between two articulating surfaces, wherein the reduction is caused, at least in part, by a lubricating agent. Lubricating properties can be measured using a variety of methods. For example, the lubricating capacity of SZP, or a fragment or derivative thereof, can be tested using a cartilage-on-glass test device, as described in greater detail in the examples below. A lubricating agent may act alone or may act by binding other molecules.

The contacting step of the methods is performed in vivo or extra-corporeally. For example, SZP, or a fragment or derivative thereof, can be injected into one or more joints of a subject to contact the articular surface or surfaces of a natural joint (i.e., non-prosthetic joint) or a prosthetic joint in vivo. Alternatively, at least in the case of a prosthetic joint, the contacting step can be performed extra-corporeally (i.e., prior to insertion into the subject).

As used herein, “a prosthetic joint” includes a joint having one prosthetic articular surface or a joint having two prosthetic articular surfaces. Thus, one or both surfaces of a prosthetic joint can be contacted with SZP or a fragment or derivative thereof, prior to insertion into the subject.

When the contacting step is in vivo, the SZP, or fragments or derivatives thereof, can be administered to achieve the desired lubricating effect in a variety of ways, known in the art, including orally, intravenously, or intra-articularly by injection into the joint. The SZP, or fragments or derivatives thereof can be administered in a carrier pharmaceutically acceptable to the subject. Suitable carriers and their formulations are described in Remington's Pharmaceutical Sciences (latest edition). Typically, an appropriate amount of a pharmaceutically-acceptable salt is used in the formulation to render the formulation isotonic. Examples of the pharmaceutically-acceptable carrier include saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the SZP, which matrices are in the form of shaped articles, e.g., films, liposomes or microparticles. It will be apparent to those persons skilled in the art that certain carriers may be more preferable depending upon, for instance, the route of administration and concentration of SZP, or fragment or derivative thereof, being administered.

Suitable carriers for oral administration of the SZP, or fragment or derivative thereof, include one or more substances which may also act as flavoring agents, lubricants, suspending agents, or as protectants. Suitable solid carriers include calcium phosphate, calcium carbonate, magnesium stearate, sugars, starch, alginate, gelatin, cellulose, carboxypolymethylene, or cyclodextrans. Suitable liquid carriers may be water, pyrogen free saline, pharmaceutically accepted oils, hyaluronan, collagen, fibronectin, albumin, or a mixture of any of these. The liquid can also contain other suitable pharmaceutical additions such as buffers, preservatives, flavoring agents, viscosity or osmo-regulators, stabilizers or suspending agents. Examples of suitable liquid carriers include water with or without various additives, including carboxypolymethylene as a pH-regulated gel. The SZP, or fragment or derivative thereof, may be contained in liposomes. Alternatively, the SZP, or fragment or derivative thereof, may be contained in enteric coated capsules that release the agent into the intestine to avoid gastric breakdown. For parenteral administration of the SZP or fragment thereof, a sterile solution or suspension is prepared in saline that may contain additives, such as ethyl oleate or isopropyl myristate, and can be injected for example, into subcutaneous or intramuscular tissues, as well as intravenously or intra-articularly. Alternatively, the SZP, or fragment or derivative thereof, can be microencapsulated with either a natural or a synthetic polymer into microparticles, which can release the SZP, or fragment or derivative thereof.

The amount of SZP, or fragment or derivative thereof, administered or the schedule for administration will vary among individuals based on age, size, weight, condition, the joint to be treated, mode of administration, and the degree of lubrication sought. One skilled in the art will realize that dosages are best optimized by the practicing physician and methods for determining dosage are described, for example in Remington's Pharmaceutical Science, latest edition. A typical dose of the SZP, or fragment or derivative thereof, might range from about 0.1 mg/kg to up to 10 mg/kg of body weight or more per day, and preferably 0.1 to up to 1 mg/kg, depending on the factors mentioned above. An intravenous injection of the SZP, or fragment or derivative thereof, for example, could be 100-4000 μg, and preferably 200-1000 μg, depending on the factors mentioned above. For injection into a joint, a typical quantity of SZP, or fragment or derivative thereof, can range from 1 μg to 1 mg. Preferably, the intrarticular injection would be at a concentration of about 10-2000 μg/ml, and preferably 100-600 μg/ml. Volumes of SZP, or fragment or derivative thereof, and carrier will vary depending upon the joint or joints to be treated, but approximately 0.5-10 ml, and preferably 1-5 ml, can be injected into a human knee and approximately 0.1 -5 ml, and preferably 1-2 ml, into the human ankle.

When the contacting step is extra-corporeal, the amount and concentration of SZP, or fragments or derivatives of SZP having lubricating properties, further depends on the type of articular surface. Thus, one skilled in the art could readily perform a lubrication test to assess the friction forces between the synthetic material composing the prosthetic surface or surfaces to determine the amount of lubrication desired. Concentrations of SZP in normal synovial fluid is about 200-400 ∥g/ml, and quantities of synovial fluid in a human knee is about 2-3 ml. One skilled in the art could assess the concentration and total volume of SZP for use in a prosthetic joint based on these normal values.

In one embodiment, the contacting step can be performed indirectly by administration of chondrocytes or other cell types containing a cDNA that encodes SZP, under conditions that promote expression of the SZP by the administered cells. More specifically, the cells could be administered by intra-articular injection. The cDNA for SZP can be introduced into the cells by any number of methods known in the art, including, for example, gene gun injection.

In one embodiment of this invention, the method of promoting lubrication of an articular surface of a joint is used, wherein the joint shows one or more signs of a degenerative condition, and more specifically, a degenerative joint condition. Thus, the invention further provides, a method of treating a subject with a degenerative joint condition or of preventing a degenerative joint condition in a subject, comprising administering to the subject a therapeutically effective amount of SZP or fragments or derivatives of SZP having lubricating properties.

“A therapeutically effective amount” is that amount that provides the desired amount of joint lubrication. Such amounts are determined as discussed above.

As used throughout, the term “degenerative joint condition” or “degenerative joint disease” includes a variety of conditions marked by inflammatory or non-inflammatory joint disease, including arthritic conditions (e.g., osteoarthritis, rheumatoid arthritis, gout, psoriatic arthritis, reactive arthritis, viral or post-viral arthritis, spondylarthritis, juvenile arthritis, synovitis, tendonitis, and systemic lupus erythematosus), CACP, osteoporosis, and trauma. Such degenerative joint diseases are characterized by morphological, compositional, and metabolic changes in articular cartilage. A subject with a degenerative joint disease may show clinical or subclinical signs of the disease, and thus demonstrate either early or late stages of the disease.

By “clinical” or “subclinical” signs is meant that the degenerative joint condition may or may not be accompanied by clinical symptoms such as pain, limited range of motion, radiologic changes in the joint, etc.

“Osteoarthritis,” as used herein, would include both primary and secondary degenerative joint disease, and a subject with osteoarthritis may show any of the early manifestations of osteoarthritis, including, for example, increased water content of the cartilage, increased collagen extractability, increased levels of annexin V, crepitus, and radiologic changes (including joint space narrowing, subchondral sclerosis or cysts, and osteophyte formation), or later manifestations, including, for example, joint pain, joint swelling, joint stiffness, reduced quality and quantity of cartilage matrix, deformity, chondrocalcinosis, and reduced range of motion.

“Rheumatoid arthritis” as used herein refers to inflammatory joint disease in both early and late stages. Signs and manifestations of the early stages can include, for example, general fatigue, joint stiffness or aching, synovial inflammation, excessive synovial fluid, joint effusion, osteoporosis in the ends of the bones forming the affected joint or joints, edematous synovial cells, and proliferation of synovial lining cells. In later stages, additional signs and manifestations can be detected, including joint pain, redness, swelling, and inflammation. Pannus can be seen in the joints. The articular cartilage surface can be eroded down to the subchondral bone. Changes in the composition of the synovial fluid can occur. Laxity in tendons and ligaments, as well as deformity, can occur and can cause limitations in joint range of motion and joint instability. Furthermore, Rheumatoid Factor(s) can be detected in the subject's blood at both early and late stages of the disease.

The invention also provides immortalized SZP-producing cells and immortalized chondrocytes derived from chondrocytes isolated from a specific zone of articular cartilage. The superficial zone protein-producing cells used to derive the immortalized cells can be selected from the group consisting of chondrocytes, synovial cells, pericardial cells, bone marrow cells, and connective tissue cells (e.g., cells from tendon, ligament, meniscus, or intervertebral disk) of any species. Preferably, the cells from which the immortalized cells of the present invention are derived are mammalian cells. Even more preferably, the mammalian cells are human cells.

By “immortalized” is meant, a cell that is capable of extended self-replication in culture, yet retains one or more properties of a normal chondrocyte or other SZP producing cell. Preferably, the immortalized cells of the present invention are as genetically and phenotypically similar to non-immortalized cell as possible. The immortalized chondrocytes, however, are unlike chondrocytes in primary culture because of the immortalized chondrocyte's capacity for extended self-replication. The immortalized chondrocytes thus retain one or more characteristics of non-immortalized chondrocytes, including, for example, producing and secreting SZP, aggrecan, biglycan, decorin, types II, IX, XI, and VI collagen, hyaluronan, link protein, and COMP. The immortalized chondrocytes preferably maintain a chondrocytic phenotype in culture, thereby expressing type II collagen and having a rounded cellular morphology. Thus, the immortalized chondrocytes preferably do not dedifferentiate into a fibroblastic phenotype by expressing type I collagen and having an extended bipolar morphology. Preferably this chondrocytic phenotype is maintained in suspension cultures (e.g., alginate or agarose), monolayer cultures, or both.

In one embodiment, the chondrocytes of the cell line express at least one property of a superficial zone chondrocyte, and, preferably, these immortalized chondrocytes express SZP or a fragment or derivative thereof. Preferably, the chondrocytes having at least one property of a superficial zone chondrocyte express collagen type II, aggrecan, and SZP or any combination thereof. In one embodiment, the SZP, or a fragment or derivative thereof, is expressed by the immortalized cell in three-dimensional culture (e.g., suspension culture). In another embodiment, the SZP or a fragment or derivative thereof is expressed by immortalized cell cultured in a monolayer.

In alternative embodiments, the chondrocytes of the cell line express at least one property of a middle zone chondrocyte or at least one property of a deep zone chondrocyte, including, for example, the absence of SZP expression or the ability to express Type II collagen or aggrecans or, in the case of middle zone chondrocyte properties, expression of CILP cartilage intermediate layer protein. See, e.g., Lorenzo, 1998. Preferably the cells derived from middle zone or deep zone chondrocytes do not express SZP. In one embodiment the immortalized chondrocytes expressing at least one property of a deep zone chondrocyte express or can be induced to express type X collagen. Immortalized chondrocytes derived from the middle or deep zones can be used as negative controls in bioasssays or methods of screening for agents that modulate SZP activity or can be used in various diagnostic and treatment methods.

The immortalized chondrocytes of the present invention are non-tumor derived. Rather, isolated chondrocytes are transformed by introducing a nucleic acid, like an oncogene, that promotes cell division and extends the lineage of the cell in culture. The chondrocytes are immortalized by transduction with a viral vector. See Condreay et al. (1999), which is incorporated herein by reference in its entirety for the method of viral transduction. For example, isolated chondrocytes can be immortalized by transducing the isolated chondrocytes with a recombinant baculovirus that encodes Simian virus 40 large T-antigen (SV40 Tag). Preferably, the recombinant baculovirus also contains one or more promoters (e.g., the cytomegalovirus immediate early promoter) and encodes a dominant selectable marker (e.g., neomycin phosphotransferase II). To select for stably transduced cells, the immortalized cells are cultured in a suitable culture medium that contains one or more substances that inhibit the growth or survival of the non-immortalized cells. For example, when the viral vector encodes neomycin phosphotransferase II, the cells are plated at low density and put under selection with the antibiotic G418. Optionally, the recombinant baculovirus also encodes a visual marker (e.g., enhanced green fluorescent protein).Preferably the immortalized chondrocytes of the present invention are not temperature sensitive.

Also provided is a method of repairing a cartilage defect comprising transplanting the immortalized chondrocytes into the region of the defect, under conditions that allow the chondrocytes to produce cartilage matrix. Preferably, the immortalized chondrocytes do not become tumorogenic.

Also provided by the present invention is a method of culturing immortalized or non-immortalized chondrocytes in serum-free medium, comprising culturing the chondrocytes in the serum-free medium supplemented with insulin, transferrin, and selenium. By “serum free” is meant that the medium, prior to the addition of the cartilage or chondrocytes, is completely devoid of serum. In one embodiment, non-immortalized chondrocytes in thin slices of articular cartilage are cultured in such serum free conditions. In an alternative embodiment, immortalized chondrocytes are cultured in such serum free conditions. Chondrocytes can be cultured under these conditions for up to 1, 2, 3, 4, 5, 6, months or more or any amount in between. Culturing chondrocytes in a serum-free medium, such as DMEM/ITS (Sigma Chem Co., St. Louis, Mo.), reduces total protein in the medium. The reduction in total protein simplifies purification of desired compounds synthesized by the chondrocytes and released into the medium. Furthermore, the purified or partially purified, compound will be free of serum contaminants. SZP, for example, released into the medium can be used in methods of treatment and imaging, without removing serum contaminants. Thus, the invention provides SZP in the absence of serum contaminants.

Thus, the invention provides methods of producing isolated SZP. One method comprises the steps of culturing immortalized or non-immortalized chondrocytes in serum-free medium under conditions that allow expression and secretion of SZP; harvesting the medium from the cultured chondrocytes; and isolating SZP from the medium. As used throughout, “isolated” means purified or partially purified. For example, isolated SZP is preferably at least about 70% pure and devoid of greater than 30% non-SZP material. Even more preferably, isolated SZP would be 75%, 80%, 85%, 90%, 95%, or 100% pure from non-SZP material.

Another embodiment of the method of making SZP is a method of making SZP, of its fragments or derivatives with lubricating properties or cell adhesion inhibiting properties, using recombinant techniques. The method comprises culturing a cell comprising an exogeneous nucleic acid that encodes the SZP or its fragment or derivative, wherein the exogeneous nucleic acid is operably linked to an expression control sequence, and wherein the culture conditions permit expression of SZP under the control of the expression control sequence; harvesting the medium from the cultured cells, and isolating the SZP from the cell or culture medium. Optionally the exogenous nucleic acid is the nucleotide sequence of SEQ ID NO: 5, or SEQ ID NO: 6, or a combination thereof. Optionally the exogenous nucleic acid sequence further encodes one or more mucin domains. In one embodiment the encoded molecule comprises an amino terminal globular domain (e.g., SEQ ID NO: 2), a mucin domain (with 1-200 mucin repeats) (e.g., SEQ ID NO: 11 or repeats thereof), and a carboxyl terminal globular domain (e.g., SEQ ID NO: 3).

In the recombinant methods, the cell can be any known host cell, including for example, a prokaryotic or eukaryotic cell. The nucleic acids that are delivered to cells, generally in a plasmid or other vector, typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

“Enhancer” generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events that trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription that may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

Those skilled in the art of molecular biology will understand that a wide variety of expression systems may be used to produce recombinant SZP polypeptides (as well as fragments, fusion proteins, and amino acid sequence variants with therapeutic angiogenic activity) for use in the methods of the invention. Thus, SZP may be produced using prokaryotic host cells (e.g., Escherichia coli) or eukaryotic host cells (e.g., Saccharomyces cerevisiae, insect cells such as Sf9 cells, or mammalian cells such as CHO cells, COS-1, NIH 3T3, or HeLa cells). These cells are commercially available from, for example, the American Type Culture Collection, Rockville, Md. (see also F. Ausubel et al., Current Protocols in Molecular Biology, John Wiley & Sons, New York, N.Y., 1998). The method of transformation and the choice of expression vector will depend on the host system selected. Transformation and transfection methods are described, e.g., in Ausubel et al., supra, and expression vectors may be chosen from the numerous examples known in the art.

A nucleic acid sequence encoding SZP is introduced into a plasmid or other vector, which is then used to transform living cells. Constructs in which a cDNA containing the entire SZP coding sequence, a fragment of the SZP coding sequence, amino acid variations of the SZP coding sequence, or fusion proteins of the aforementioned, inserted in the correct orientation into an expression plasmid, may be used for protein expression. In some cases, for example, when an SZP polypeptide is to be produced directly within a patient's cells, it may be desirable to express the SZP coding sequence under the control of an inducible or tissue-specific promoter.

Eukaryotic expression systems permit appropriate post-translational modifications to expressed proteins. Thus, eukaryotic, and more preferably mammalian expression systems, allow glycosylations patterns comparable to naturally expressed SZP. Transient transfection of a eukaryotic expression plasmid allows the transient production of SZP by a transfected host cell. SZP may also be produced by a stably-transfected mammalian cell line. A number of vectors suitable for stable transfection of mammalian cells are available to the public (e.g., see Pouwels et al., Cloning Vectors: A Laboratory Manual, 1985, Supp. 1987), as are methods for constructing such cell lines (see e.g., F. Ausubel et al., Current Protocols in Molecular Biology, John Wiley & Sons, New York, N.Y., 1998). Another preferred eukaryotic expression system is the baculovirus system using, for example, the vector pBacPAK9, which is available from Clontech (Palo Alto, Calif.). If desired, this system may be used in conjunction with other protein expression techniques, for example, the myc tag approach described by Evan et al. (Mol. Cell Biol. 5:3610-3616, 1985) or analogous tagging approaches, e.g., using a hemagluttinin (HA) tag.

Once the recombinant protein is expressed, it can be isolated from the expressing cells by cell lysis followed by protein purification techniques such as affinity chromatography. In this example, an antibody that specifically binds to SZP, which may be produced by methods that are well-known in the art, can be attached to a column and used to isolate SZP. Once isolated, the recombinant protein can, if desired, be purified further, e.g., by high performance liquid chromatography (HPLC; e.g., see Fisher, Laboratory Techniques In Biochemistry And Molecular Biology, Work and Burdon, Eds., Elsevier, 1980).

Using recombinant techniques for making SZP, or fragment or derivative thereof, the isolated SZP, fragment or derivative, optionally lacks glycosylation. Such an SZP lacking glycosylation preferably has a molecular weight of about 110 kDa, 120 kDa, 130 kDa, 140 kDa, or any amount in between. In other embodiments, the isolated SZP or fragment or derivative is glycosylated, preferably having a molecular weight of greater than 280 kDa, 290 kDa, 300 kDa, 310 kDa, 320 kDa, 330 kDa, 340 kDa, 350 kDa, or any amount in between. Recombinantly produced SZP, in its glycosylated or non-glycosylated form, or its fragments or derivatives can be used in all of the methods disclosed herein.

The invention also provides a method of imaging an articular surface or synovium of a joint, comprising contacting the articular surface or synovium of the joint with detectably tagged SZP or a detectably tagged SZP binding protein, under conditions in which the detectably tagged SZP or binding protein binds to the articular surface or synovium, and visualizing the detectable tag in a plurality of locations on the articular surface or synovium. Visualization of the detectable tag shows the articular surface or synovium of the joint. A “detectable tag” is any tag that can be visualized with imaging methods. The detectable tag can be a radio-opaque substance, radiolabel, a fluorescent label, or a magnetic label. The detectable tag can be selected from the group consisting of gamma-emitters, beta-emitters, and alpha-emitters, gamma-emitters, positron-emitters, X-ray-emitters and fluorescence-emitters suitable for localization. Suitable fluorescent compounds include fluorescein sodium, fluorescein isothiocyanate, phycoerythrin, and Texas Red sulfonyl chloride. See, de Belder & Wik (1975). Those skilled in the art will know, or will be able to ascertain with no more than routine experimentation, other fluorescent compounds that are suitable for labeling SZP.

Suitable radioisotopes for labeling SZP include Iodine-131, Iodine-123, Iodine-125, Iodine-126, Iodine-133, Bromine-77, Indium-111, Indium-113m, Gallium-67, Gallium-68, Ruthenium-95, Ruthenium-97, Ruthenium-103, Ruthenium-105, Mercury-107, Mercury-203, Rhenium-99m, Rhenium-105, Rhenium-101, Tellurium-121m, Tellurium-122m, Tellurium-125m, Thulium-165, Thulium-167, Thulium-168, Technetium-99m and Fluorine-18.

The visualization step can comprise a means of visualization selected from the group consisting of nuclear magnetic resonance, radioimmunoscintigraphy, X-radiography, positron emission tomography, computerized axial tomography, magnetic resonance imaging, and ultrasonography. For visualization, the subject, for example, can be scanned with a gamma ray emission counting machine such as the axial tomographic scanner commercially available under the designation CT (80-800 CT/T) from General Electric Company (Milwaukee, Wis.), or with a positron emission transaxial tomography scanner.

The gamma-emitters Indium-111 and Technetium-99m can be detected with a gamma camera and have favorable half lives for imaging in vivo. The SZP, for example, can be labeled with Indium-111 or Technetium-99m via a conjugated metal chelator, such as DTPA (diethlenetriaminepentaacetic acid). See Krejcarek et al. (1977); Khaw et al. (1980); Gansow et al., U.S. Pat. No. 4,472,509; Hnatowich, U.S. Pat. No. 4,479,930, the teachings of which are incorporated herein by reference.

For purposes of imaging the articular surface or synovium, the SZP or binding proteins can be administered by a variety of techniques discussed above.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention. These examples are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Isolation and Purification of Human Superficial Zone Protein (SZP) from Cartilage

Human tali were obtained, through collaboration with The Regional Organ Bank of Illinois (ROBI) with the approval of the institutional review board (IRB) of the Medical College of Rush Presbyterian St. Luke's Medical Center, from cadaveric organ donors within 24 hours of death. Individual entire human tali were submerged in approximately 100-130 ml of medium consisting of Dulbecco's Modified Eagle Medium (DMEM) supplemented with 5% fetal bovine serum, 25 μg/ml ascorbic acid and 20 μCi of ³H-proline for 18-22 hours in a humidified atmosphere of 5% CO₂/air at 37° C. with constant stirring. After the incubation period, the medium (containing ³H-proline labeled Superficial Zone Protein (SZP) was harvested and six Complete™ mini protease inhibitor cocktail tablets (Boehinger Mannheim, Gmbh. Germany) were added. Dry guanidinium hydrochloride (GuHCl) was added to the medium to bring the concentration of GuHCl to 4 M. The solution was brought to an initial density of 1.46 gm/ml by the addition of Cesium chloride (0.57 grams per gram of medium). The solution was subjected to equilibrium density gradient ultracentrifugation at 33,000 RPM for 40 hours at 10° C. The resulting gradient was fractionated into five equal portions, designated as D5 at the top to D1 at the bottom of the gradient solution. The D5 fraction was dialyzed against water and brought to 8 M urea, 0.005 M EDTA, 0.15 M sodium chloride, 0.05 M sodium acetate, pH 6.0 by the addition of dry chemicals and acetic acid. This solution was subjected to anion exchange chromatography on DEAE Sephacel equilibrated in 8 M urea, 0.15 M sodium chloride, 0.005 M EDTA, 0.05 M sodium acetate at pH 6.0 and the SZP was eluted from the DEAE in a stepwise fashion using increasing concentrations of sodium chloride of 0.3 M and 0.6 M salt. SZP eluted between 0.3 and 0.6 M sodium chloride. The SZP containing fraction was dialyzed against water, lyophilized, dissolved in column buffer and subjected to column chromatography on Sepharose CL-4B in the presence of 4 M GuHCl, 0.1 M sodium sulfate, 0.005 M EDTA and 0.05 M sodium acetate, pH 5.8. The eluate from the column was collected in equal fractions and the fractions were analyzed for the presence of ³H-proline by scintillation counting. Putative SZP containing fractions were pooled, dialyzed against water, lyophilized, dissolved in sample buffer and analyzed by SDS-PAGE to confirm the presence of SZP. This entire procedure was repeated without the presence of ³H-proline and the final lyophilized material was used as antigen for the production of monoclonal antibodies.

Using the protocol outlined above, enough SZP was obtained to immunize several mice. The SZP had a molecular weight of approximately 345,000 Daltons by SDS-PAGE and no other bands were observed.

### Example 2

### Isolation and Purification of Human Superficial Zone Protein (SZP) from Media of Cultures of Chondrocytes and from Synovial Fluid

SZP was purified from culture medium or synovial fluid by a combination of affinity chromatography, first on a peanut lectin and then on a monoclonal anti-SZP antibody column. Culture medium or synovial fluid was made 0.5 M in NaCl and 5 mM in EDTA and clarified by centrifugation at 10,000 g for 15 minutes. The supernatant, either 50 ml of culture medium or 5 ml of synovial fluid, was incubated with 5 ml of peanut lectin-agarose beads (Sigma, St. Louis, Mo.) with rotation overnight at 4° C. The lectin beads were washed with 25 ml of 10 mM sodium phosphate, 0.5 M NaCl, 5 mM EDTA, pH 7.5 buffer. The bound SZP was eluted with the same buffer containing 0.4 M lactose. The lectin beads were subsequently washed with the washing buffer containing 1.5 M NaCl and then again with washing buffer containing 0.35 M lactose and 1.5 M NaCl. The majority of the SZP was eluted in the first elution with 0.4 M lactose. This SZP preparation also contained small amounts of fibronectin and albumin.

The SZP preparations were further purified on an anti-SZP monoclonal antibody affinity column. Five ml of Sepharose CL-2B (Sigma, St. Louis, Mo.) was activated with CNBr as described by March et al. (1974)) and incubated with 2.5 mg each of purified monoclonal antibodies GW6.79 and 17.106. Residual reactive sites were blocked with 0.1 M Tris, pH 9.8 for 1 h and the beads washed with 2 M urea, followed by 1 M NaCl in PBS buffer. Antibody conjugation efficiency to the Sepharose beads was greater than 80%. SZP preparations were made 1 M in NaCl and 1% Triton and incubated with the anti-SZP beads overnight with rotation at 4° C. The beads were washed with PBS containing 1 M NaCl and 1% Triton. The bound SZP was eluted with 2 M guanidine hydrochloride, pH 7.5. The eluted SZP was dialyzed against 0.5 M NaCl, 10 mM sodium phosphate, pH 7.5 and stored at −20° C. These preparations yielded a single band of SZP at 345 kDa by SDS-PAGE.

### Example 3

Production of Monoclonal Antibodies to SZP

A. SZP Immunization

Purified human SZP, which was prepared as described in Example 1, was used as the antigen for immunization for antibody production. Two 8 week old female SJL mice were immunized using either a RIMMS (Repetitive Immunization Multiple Sites) protocol or a conventional immunization regime (e.g., Su et al, 1999). For RIMMS, one 8-week-old female SJL mouse (Jackson Laboratories, Bar Harbor, Me.) was immunized on days 0, 3, 5, 7 and 11, following the RIMMS immunization regime (Kilpatrick et al., 1997). The mouse was anesthetized with isofluorane prior to each series of immunizations. Twelve sites proximal to the draining lymph nodes were injected subcutaneously with 50 μl per site of antigen/adjuvant mixture. Six of the sites received antigen diluted 1:1 with complete Freund's adjuvant (FCA, GibcoBRL) and the six juxtaposed sites received antigen diluted 1:1 in RIBI adjuvant (RIBI Immun. Chem. Research, Inc., Hamilton, Mont.).

One eight week old female SJL mouse was immunized, using a conventional immunization regimen (e.g., Su et al, 1999), on day 0, 14, 21, and 24. The immunizations on day 0 and 14 were I.P. with the antigen diluted 1:1 in RIBI adjuvant. The day 21 immunization was I.V. with the antigen diluted in sterile PBS. The final immunization was I.P. with the adjuvant diluted in sterile PBS.

B. PEG Induced Somatic Fusion Protocols

Mice were sacrificed, and a single cell suspension was prepared from either the spleen or the lymph node cells (brachial, axillary, superficial inguinal and popliteal). These cells were combined at a ratio of 2.5:1 with the modified myeloma cell line P3XBcl-2-13 (Kilpatrick et al., 1997). Somatic fusion was performed using 1 ml of 50% polyethylene glycol 1500 (Boehringer Mannheim, GmbH, Germany). Pelleted cells were resuspended in media containing an equal volume of Excell-610 (JRH Biosciences, Lenexa, Kans.) and RPMI 1640 supplemented with 10% FBS, 10% Origen Cloning Factor (Igen, Rockville Md.), 2 mM L-glutamine, 100 μg/ml penicillin, and 0.01 mM 2-ME; plated out at 1 ml per well in 24 well plates; and incubated overnight at 37° C. After 24 hours, 1 ml of selection media containing a 2× concentration of HAT (0.1 mM hypoxanthine, 0.16 mM thymidine, and 4 mM aminopterin, GibcoBRL) prepared in the above media, was added to each well. After one week in culture, media was changed to contain HT (0.1 mM hypoxanthine, 0.16 mM thymidine, GibcoBRL).

### Example 4

Screening of Potential Monoclonal Antibodies to SZP

A. Primary ELISA Screening

Aggrecan isolated from bovine nasal cartilage (BNS) and rat chondrosarcoma (RCS), which served as negative controls, were resuspended in 0.1M Tris pH 8.0, aliquoted 1 ml/vial, and frozen at −80° C. The plates were then coated with 3 μg/ml of either SZP, BNS, or RCS. Antigen was diluted in carbonate coating buffer, pH 9.2-9.6, and plated 100 μl/well on Costar 96 well assay plates (Corning, N.Y.). The plates were incubated for 2 hr at 37° C. Then blocked with 100 μl/well of TBS containing 5% normal goat serum and lmg/ml PEG for 30 min at 37° C. 100 μl/well of tissue culture media corresponding to each individual well of the fusion plates were added and incubated for 1 hr at 37° C. The plates then were washed 3× with 200 μl/well of 1× TBS+1% Tween 20. 100 μl/well of secondary antibody (goat anti-mouse IgG-alkaline phosphatase conjugate) diluted 1:1000 in blocking buffer were added. Plate was then incubated for 1 hr at 37° C. The plates were then developed with Sigma 104 phosphatase substrate and the color change was read at 15 and 30 min.

All wells were tested for binding, and all were growth positive. Seven wells were selected as positive from the conventional immunization and fusion. These seven wells were plated at 30 cells per plate for limiting dilution cloning. Four wells from the RIMMS fusion that were selected as positive were also plated to clone by limiting dilution. Aliquots of the supernatants were further analyzed in the subsequent assays.

B. Antibody Staining of Chondrocyte Cultures

Thin slices of articular cartilage from human tali were manually dissected from the superficial, middle and deep zones of the cartilage and placed in DMEM. The slices from the middle zone were discarded. The cartilage slices from the superficial and deep zones were treated separately with 0.2% pronase in DMEM supplemented with 5% fetal bovine serum for 1.5 hours at 37° C. (Aydelotte and Kuettner (1988); Aydelotte et al. (1988); Schumacher et al. (1999)). The slices were then rinsed extensively with DMEM and treated further with 0.025% Collagenase P for 18-22 hours in DMEM supplemented with 5% fetal bovine serum. The resulting chondrocyte suspensions were centrifuged at 1000 RPM for 15 minutes in order to pellet the cells. The chondrocytes were washed in DMEM three times and centrifuged as stated above to collect the cells. The number of chondrocytes in each sample was determined by counting the cells on a hemocytometer. Chondrocytes from the superficial and deep zones were seeded separately into a 96 well tissue culture plate at high density (250,000 cells/cm²) in medium consisting of DMEM supplemented with 10% fetal bovine serum. The cells were allowed to attach overnight and refed with medium consisting of DMEM supplemented with 10% fetal bovine serum and 25 μg/ml ascorbic acid. After three days in culture the cells were refed with medium plus 10⁻⁶ M monensin for four hours in order to prevent secretion through the Golgi apparatus. At the end of the incubation period the cells were rinsed briefly in phosphate buffered saline (PBS) and fixed with a solution of 4% paraformaldehyde in PBS, pH 7.4 for five minutes at room temperature. The cells were rinsed in PBS and permeabilized with a solution of 0.1% Triton-X 100® (Sigma Chemical Co., St. Louis, Mo.) for five minutes at room temperature. The cells were rinsed in PBS and non-specific binding sites were blocked with a solution of 1% bovine serum albumin (BSA) and 1% normal goat serum for 20 minutes at room temperature. The cells were rinsed in PBS and pairs of wells containing cells from the superficial and deep zones were incubated with different hybridoma media, potentially containing a monoclonal antibody to SZP for 45 minutes at room temperature. The cells were rinsed in PBS and incubated with a goat anti-mouse rhodamine conjugated IgG diluted 1:50 with PBS for 45 minutes at room temperature. The cells were rinsed in PBS and examined by fluorescence microscopy. Any pair of wells containing cells from the superficial and deep zone that was positive in the chondrocytes from the superficial zone and negative in the chondrocytes from the deep zone was considered as a positive reaction as a monoclonal antibody to SZP. Four monoclonal antibodies that were positive for the superficial chondrocytes and negative for the deep chondrocytes were obtained. They were designated as GW 3.15, GW 4.10, GW 4.23 and GW 5.15.

C. Direct ELISA for Human SZP

A 96 well ELISA plate was coated overnight at 4° C. with conditioned media from human talar superficial chondrocytes or deep chondrocytes in the presence of 20 mM NaHCO₃/Na₂CO₃, pH 9.2. All wells were rinsed and incubated with the various hybridoma media for 1 hour at room temperature. The wells were rinsed and incubated with a horseradish peroxidase conjugated goat anti-mouse IgG for 1 hour at room temperature. The wells were rinsed and color development was achieved using hydrogen peroxide and o-phenylenediamine as the chromogenic substrate. Plates were read with an automatic ELISA plate reader. Any pair of wells containing conditioned media from chondrocytes from the superficial zone and chondrocytes from the deep zone in which there was a positive result for the superficial chondrocytes and not the deep chondrocytes was considered positive for SZP.

This method was also used to test samples of human synovial fluids from normal donors and patients with osteoarthritis (OA) and rheumatoid arthritis (RA). Direct ELISA of samples of synovial fluids from normal donors, patients with osteoarthritis and patients with rheumatoid arthritis revealed that SZP is elevated in synovial fluids from patients with OA or RA compared to normal synovial fluid.

D. Immunohistochemistry of Human Knee and Ankle Cartilage

Samples from full thickness slices of cartilage and thin slices from the superficial zone from the articular surface from human femoral condyle and talar dome cartilage were obtained within 24 hours of the death of the donor. Cartilage samples were fixed in 4% paraformaldebyde/PBS for 30 minutes at room temperature and rinsed in PBS. Vertical frozen sections and paraffin embedded sections were obtained from samples of the full thickness of cartilage from human knee and ankle cartilages. Horizontal frozen sections and paraffin embedded sections were obtained from the thin slices of cartilage from the superficial zone. Some cartilage samples were pre-treated with monensin at a concentration of 10⁻⁶ M for four hours before fixation. Sections of cartilage were rinsed in PBS, permeabilized in 0.1% Triton-X 100® (Sigma Chem. Co., St. Louis, Mo.) for five minutes at room temperature and rinsed in PBS. Non-specific binding sites were blocked in a solution of 1% BSA, 1% normal goat serum for 20 minutes at room temperature. The sections were rinsed in PBS and incubated with the monoclonal antibody GW 4.23 (mAb GW 4.23) for 45 minutes at room temperature. The sections were rinsed in PBS and incubated with a horseradish peroxidase conjugated goat anti mouse IgG for 45 minutes at room temperature. The sections were rinsed in 0.05 M Tris, pH 7.6 and positive SZP sites were visualized using hydrogen peroxide and diaminobenzidine as the chromogenic substrate. Alternatively, sections for immunohistochemistry were tested using the Pierce Immunopure® ABC Alkaline Phosphate mouse IgG staining kit (Pierce, Rockford, Ill.), following all manufacturers directions.

Using mAb GW 4.23, the chondrocytes in the superficial zone of articular cartilage from knee and ankle samples were positive for SZP whereas the chondrocytes in the middle and deep zones were non-reactive. A thin layer of immuno-positive material for SZP was also observed at the articular surface in vertical sections of articular cartilage from both knee and ankle samples. Horizontal sections of the superficial zone also revealed a fine meshwork of immuno-positive material for SZP at the articular surface.

### Example 5

SDS-PAGE and Western Blotting

SDS-PAGE was performed on 4-10% gradient separating gels, with a 3.6% stacking gel. Samples for SDS-PAGE were dissolved in sample buffer consisting of 1% SDS, 0.08 M Tris, pH 6.8 containing 16% ethylene glycol and 0.0006% bromophenol blue. All samples were run non-reduced and not boiled. Separated proteins were transferred to nitrocellulose by Western blotting. Western blotting was performed overnight at 250 mAmps in a buffer consisting of 12 mM Tris, pH 7.4, 0.03 mM EDTA and 6 mM sodium acetate. Non-specific binding sites on the nitrocellulose membrane containing the separated proteins were blocked in a solution of 5% non-fat milk in PBS for 30 minutes at room temperature and rinsed in PBS. The nitrocellulose membrane was incubated with mAb GW 4.23 (1:10 dilution) for 1 hour at room temperature. The membrane was rinsed in PBS and incubated with a horseradish-peroxidase conjugated goat anti mouse IgG (1:500 dilution) for 1 hour at room temperature. The membrane was rinsed in 0.5 M Tris pH 7.6 and protein bands specific for the epitope recognized by mAb GW 4.23 were visualized using hydrogen peroxide and 4-chloro-1-napthol as the chromogenic substrate.

SDS-PAGE of purified SZP revealed a single band with an apparent molecular weight of 345,000 Daltons compared to globular standards. Samples of conditioned medium from slices of the superficial zone from knee and ankle cartilage showed a protein band identical to purified SZP.

### Example 6

Staining of the Articular Surface of Human Tali using GW 4.23

Several cylindrical punches of the full thickness of articular cartilage, 8 mm in diameter, were obtained from the talar dome of human ankles. These cartilage plugs were fixed in 4% paraformaldehyde/PBS for 30 minutes at room temperature. The plugs were rinsed in TBS. Non-specific binding sites were blocked with 1% BSA, 1% NGS for 20 minutes at room temperature in TBS. The GW 4.23 antibody was applied to different plugs for different amounts of time. Time points of 0, 5, 15, 30, 60, and 120 minutes were used. The plugs were rinsed in TBS and a biotinylated second antibody and avidin-biotin alkaline phosphatase complex applied as stated above for immunohistochemistry. The plugs were incubated with NBT/BCIP substrate used at half strength until maximal color development was achieved. Thirty minutes was chosen as the optimal time of incubation of the first antibody with the cartilage samples and was used for subsequent experiments.

In a different experiment, a matched pair of intact human ankle joints was obtained and one ankle was injected with GW 4.23 (1:10 dilution in DMEM) into the synovial cavity and the other ankle was injected with DMEM. Both joints were incubated for 30 minutes at 37° C. in a humidified chamber. At the end of the incubation times both joints were opened and cylindrical punches of the full thickness of articular cartilage were taken. The punches were fixed in 4% paraformaldehyde/PBS for 30 minutes at room temperature. The punches were rinsed extensively in PBS and then processed as stated above for immunohistochemistry. The punches were placed in NBT/BCIP used at half strength until maximal color development occurred.

In another experiment, a matched pair of normal intact human ankle tali (Collin's grade 0) was obtained and one talus was fixed as stated above and incubated with GW 4.23 (1:10 dilution) for 30 minutes at room temperature. The other talus was fixed and incubated with mouse IgG used at the same concentration of GW 4.23. Both tali were processed as stated above for immunohistochemistry. The tali were placed in NBT/BCIP used at half strength until maximal color development was achieved.

One talus with degenerative changes was obtained (Collin's grade 2). This talus had fissures and a small lesion off to one side of the talar dome. The talus was fixed and processed as stated above using GW 4.23 (1:10 dilution). The talus was placed in NBT/BCIP used at half strength until maximal color development was achieved.

A large piece of cartilage from femoral condyle removed during knee replacement surgery was obtained. The sample was fixed as stated above and processed as stated above using GW 4.23 (1:10 dilution). The sample was placed in NBT/BCIP used at half strength until maximal color development was achieved.

In the experiments outlined here, Mab GW 4.23 was used successfully to stain the articular surface of cylindrical plugs of articular cartilage from human tali. Staining of the articular surface was present at all time points of incubation of Mab GW 4.23 with the tissue samples from as little as 5 minutes incubation with the antibody to as long as 2 hours incubation with the antibody with the optimal time of incubation being 30 minutes. There was no staining of the cartilage when the Mab GW 4.23 was omitted (time point 0). In these experiments, only the surface of the cartilage plug was stained. No staining was seen in the cells or at the deep cut end of the cartilage plug.

Positive staining of the articular surface of cylindrical cartilage plugs was observed when Mab GW 4.23 was injected into an intact human ankle joint prior to fixation or processing of the tissue. No staining was seen in the cells or at the cut edges of the cartilage plug.

Positive staining of the surface of normal human intact ankle tali was observed when Mab GW 4.23 was used in these experiments. The staining at the articular surface was smooth and even and showed no defects at the articular surface. There was also positive staining observed in the synovial tissue surrounding the cartilage. There was no staining at the articular surface of the tali that was treated with the same concentration of a non-specific IgG control.

The articular surface of a human talus showing degenerative changes showed uneven heterogeneous staining when Mab GW 4.23 was used to stain the tissue. Fissures in the articular surface were stained darker than surrounding tissue and areas were present at the surface which were unstained. The lesion site showed very dark staining.

When Mab GW 4.23 was used to stain a piece of cartilage removed from a patient undergoing joint replacement, the surface stained intensely even though there was no superficial zone present. Upon closer examination of the tissue it was found that the staining was due to material deposited at the surface of the damaged cartilage. There was no detectable cellular staining or staining of the matrix within the tissue. All the staining material was at the damaged surface.

Collectively, these experiments demonstrate that a monoclonal antibody to the superficial zone protein can successfully be used to visualize the surface of articular cartilage in both normal and damaged joints as well as the surrounding synovial tissue.

### Example 7

Preparation of Proteolytic SZP Fragments and Assignment of Epitope-Containing Domain

A modification of the method of Su et al. (1995) is used to assign the epitope-containing domain of SZP. Purified SZP is reduced and alkylated by incubating the protein in 6M guanidine-HCl, 0.5 M Tris-HCl, 10 mM EDTA and 20 mM dithiothreitol (pH 8.6) for 1 h at 37C. under nitrogen, followed by addition of 4-vinylpyridine to 50 mM for 30 min at room temperature. The pyridylethylated material is desalted by HPLC with a BU300 column (2.1×30 mm, Brownlee, Foster City, Calif.) using a linear gradient of acetonitrile (16-64%) in 0.1% trifluoroacetic acid (TFA) over 30 min. The eluted protein is then digested with sequencing grade Lys-C (Wako, Richmond, Va.) in 0.1M Tris-HCl (pH 8.5) for 16 h at room temperature, with an enzyme:substrate ratio of 1:100. The Lys-C generated peptides are then separated and isolated on the BU300 column using a linear gradient of acetonitrile (8-64%) in 0.1% TFA over a 40 min period. Peptide fragments are dried by flushing with nitrogen and are then resuspended in TBS. Automated Edman degradations are performed using the Applied Biosystems 477A liquid-pulse sequencer (Applied Biosystem, Foster City, Calif.) equipped with a 120A PTH analyzer for the identification of phenylthiohydantoin amino acids.

The SZP protelytic fragments separated by HPLC are used for coating an ELISA plate for reaction with anti-SZP antibody. The fragment that is recognized by anti-SZP is identified as the epitope-containing domain.

### Example 8

Antibody Affinity as Determined by BIAcore Analysis

BIAcore technology and its use in characterizing inter-molecular interactions has previously been described (Fägerstam et al. (1992)). The BIAcore 2000 system, CM5 sensor chips, P-20 surfactant, the coupling kit which contained N-hydroxysuccinimide, N-ethyl-N′-(3-diethylaminopropyl)-carbodiimide, ethanolamine hydrochloride pH 8.5, and rabbit anti-mouse FC-γ is from Pharmacia Biosensor AB (Uppsala, Sweden). All other chemicals are reagent grade. The BIAcore running buffer used for immobilization and binding studies contains 10 mM HEPES (pH 7.4), 150 mM NaCl, 0.05% volume of a 10% P-20 surfactant solution.

Carboxyl groups of the BIAcore CM5 sensor chip hydrogel matrix is activated for 7 min with a mixture of 50 mM N-hydroxysuccinimide and 200 mM N-ethyl-N′-(3-diethylaminopropyl)-carbodiimide. Rabbit anti-mouse Fc-γ (RAMfc) antibody is diluted to 40 μg/ml in 10 mM sodium acetate pH 5.0 then is injected onto the sensor chip for 3 min at a flow rate of 5 μl/min. Unreacted groups are then deactivated with a 7-min injection of 1 M ethanolamine hydrochloride pH 8.5. To determine binding constants, antibodies are injected over the RAMfc at 5 μl/min. for 4 min. The flow is then increased to 40 μl/min and dilutions of human SZP or bovine SZP are injected for 1 min. The surface is regenerated with 100 mM HCl. Binding constants are determined using BIAevaluation software.

### Example 9

Quantitation of SZP Using Homogenous Formats

A. Scintillation Proximity Assay (SPA)

SZP antibody, radiolabeled (beta emitter) SZP or SZP fragment and the scintillant-embedded polyvinyl toluene beads conjugated with anti-mouse or protein A are mixed together. When radiolabeled SZP or SZP fragment captured by anti-SZP, the beta emitter are brought to the proximity of scintlillant-embedded beads, resulting in the emission of light that can be measured by a scintillation counter.

B. Homogeneous Time-Resolved Fluorescence Assay (HTRFA)

Biotinlyted SZP or SZP fragment, lanthanide chelate-labeled anti-SZP (fluorescence energy donor) and streptavidin conjugated with the energy acceptor are incubated together to allow the energy donors to be in the proximity of energy acceptors. Donor/acceptor pairs may include for example, Eu/allophycocyanin (or Cy5) or Terbium (Tb)/tetramethylrhodamine. Upon excitation of the donor, the specific energy is transferred from the donor to the acceptor, and the resultant fluorescent signals can be measured by a time-resolved fluorometer.

C. Fluorescence Polarization Assay (FPA)

Fluorescent labeled SZP fragment (<30 kDa) and SZP antibody are mixed together to allow the antibody binding to SZP fragment. After the binding reaches equilibrium, the immune complex, due to increased in mass, tumbles more slowly, thus, yielding a polarized fluorescence signal that can be measured by a fluorescent polarization meter. Alternatively, Fab fragment of the SZP monoclonal antibody can be fluorescent labeled and an increase in polarization can be monitored with binding to SZP.

### Example 10

DNA-Based Immunization for the Production of SZP Monoclonal Antibodies

DNA plasmid preparation, DNA/gold particle bullets and delivery of DNA bullets to mouse epidermis have previously been reported (Kilpatrick et al. (1998); Eisenbraun et al. (1993); Pertmer et al. (1996)). DNA encoding the N- or the C-terminal region of SZP is cloned into the Alpha+vector that has human Fc cDNA inclusion (Kaplan et al. (1997)). The Alpha+SZP/Fc plasmid is transfected into E. coli and DNA is prepared from a selected clone. After DNA/gold particle bullets are prepared, DNA/gold particles are propelled into the shaved thorastic and abdominal regions of mice using a helium-driven Accell gene gun (PowerJet Vaccines, Incorp. 585 Science drive, Suite C, Madison, Wis. 53711). Following the primary immunization, mice receive one to four booster immunization/s within 8-11 days. On the day of fusion (day 9-13), lymphocytes harvested from axillary, brachial and superficial inquinal nodes are prepared and fused with myeloma cells following a previously published protocol (Su et al (1999)).

### Example 11

Immunization via Recombinant Baculovirus Displaying SZP-Fusion Proteins for the Production of SZP Monoclonal Antibodies

A. Generation of SZP Fusion Transfer Plasmids

The Baculovirus fusion protein is produced using the BacVector Virus Display system from Novagen (Madison, Wis.). Cloning, subcloning and sequencing of DNA are carried out using standard protocols (Sambrook et al. (1989)). The amino-terminal domains of human SZP are amplified and cloned into the Kpn I site of the pBACsurf I vector. Positive plaques are selected based on anti-gp64 staining of both native gp64 and gp64 fusion bands in Western blot analysis (Lindley et al. (2000). The virus is then scaled up to a 150 ml suspension culture (1×10⁶ cells/ml), and incubated on a shaker for 3 days at 27° C. For generation of antigen for immunizations, 450 ml of Sf9 cells at 1×10⁶ cells/ml are infected with relevant virus, at a multiplicity of infection (MOI) of 0.1, and grown for 3 days at 27° C. To harvest virus, the culture supernatant is cleared by high-speed centrifugation for 3 hr at 61,000×g. The virus pellet is resuspended in phosphate buffered saline (PBS) and filtered through a 0.2 μM filter. Virus is diluted in PBS and the mice are immunized as described in Example 3 using the RIMMS immunization regime detailed below. The total amount of antigen used for immunizations is approximately 15 μg of the SZP.

B. ELISA Screening

Primary ELISA screenings are performed using previously published procedures (Harlow & Lane 1988). High binding EIA plates (Corning/Costar Corning, N.Y.) are coated with whole cell lysates from Sf9 cells infected with either a control virus, or the SZP-fusion virus to allow subtractive comparisons. Lysates are prepared from cells infected at a multiplicity of infection (MOI) of 1 plaque-forming unit (pfu)/cell at 48 hr post infection. Infected cells are pelleted and subjected to repeated freeze-thaw cycles in a dry ice ethanol bath. The lysates are then diluted 1:10 in carbonate coating buffer and 100 μl per well are incubated at 37° C. for 1 hr. Plates are blocked with 100 μl/well of Tris buffered saline (TBS), containing 5% normal goat serum and 1% polyethylene glycol (PEG) for 1 hr at 37° C. Undiluted tissue culture supernatant is added at 100 μl/well and incubated at 37° C. for 1 hr. Plates are washed with 1× TBS+1% Tween 20 (TBS-T). Secondary antibody, goat anti-mouse IgG-alkaline phosphatase conjugate light chain specific (Southern Biotechnology Associates, Birmingham Ala.), was diluted 1:1000 in blocking buffer, and 100 μl/well is reacted for 1 hr at 37° C. Plates are developed with phosphatase substrate (Sigma, St. Louis, Mo.) at room temperature and readings are taken at 15 and 30 minutes. SZP reactive supernatants are further characterized as described in examples above.

### Example 12

Immunolocalization of SZP in Human Articular Cartilage

Eight μm vertical frozen sections of normal (Collin's Grade 0) human cartilage taken from the femoral head and talar cartilage were stained with the antibody to SZP as described above. The tissue was treated with monensin prior to sectioning in order to prevent secretion of SZP from the chondrocytes. There is a positive stain in the flattened chondrocytes in the superficial zone. In the absence of treatment with monesin, there was intense staining for SZP at the articular surface.

Horizontal frozen sections (“en face”) taken from the articular surface of human tali also were examined. These samples were treated with monensin to prevent secretion of SZP from the chondrocytes. SZP-positive staining material was observed above the chondrocytes at the articular surface, in a reticular type of pattern. Clusters of chondrocytes in the superficial zone stained positive for SZP. Chondrocytes deeper in the tissue were negative for SZP.

Eight mm vertical frozen sections of human synovium were also examined. The cells lining the synovial villus stained positively for SZP.

Additional cartilage samples from normal human ankle and knee were fixed in 4% paraformaldehyde for 30 minutes and processed for paraffin embedding and sectioning. Similar results were obtained as for frozen sections; SZP positive cells were visualized in the superficial zone of the cartilage but not the middle or deep zones.

### Example 13

Purification of Cartilage SZP from Chondrocytes Cultured in Serum-Free Conditions

Thin slices from the superficial zone of the talar dome from human ankle or from the femoral head of human knee were manually dissected and immediately placed in Dulbecco's Modified Eagle Medium (DMEM) (Gibco BRL, Life Technologies, Gaithersburg, Md.) until all tissue was collected. The cartilage slices were then transferred to a spinner flask with either DMEM/ITS (insulin, transferrin, selenium) (Sigma Chem. Co., St. Louis, Mo.) or DMEM/5% FBS (fetal bovine serum, Hyclone Laboratories, Logan Utah). The cartilage slices were cultured in a humidified atmosphere of 5% CO₂/air at 37° C. This medium was chosen to avoid contamination of the cartilage-derived SZP with serum proteins and serum-derived SZP. Radiolabeling experiments and sandwich ELISA assays for SZP showed the cartilage slices remained viable for more than two months. Cultures maintained in the presence of insulin, transferrin and selenium synthesized about 90% of the amount of SZP as did similar cultures maintained in DME plus 10% fetal calf serum.

Culture medium was harvested every two days and the cartilage slices were refed three times per week. EDTA was added to the harvested culture media to a final concentration of 0.005 M and the media were frozen at −20° C. until farther processed. Cultures were maintained for two months.

Frozen media were thawed at room temperature, pooled and made 8 M urea, 0.05 M sodium acetate, pH 6.0 by the addition of dry chemicals and acetic acid. This solution was subjected to anion exchange chromatography on DEAE Sephacel™ (Amersham Pharmacia Biotech AB, Uppsala, Sweden) in a batch-wise manner. Two hundred ml of washed DEAE gel was slowly stirred with 2 liters of media solution at 4° C. overnight. Adsorbed molecules were eluted with increasing concentrations of sodium chloride of 0.015 M, 0.3 M, 0.6 M and 2.0 M NaCl by vacuum filtration. These different pools of eluted material were tested for the presence of SZP by dot blot analysis. The pool most enriched in SZP was dialyzed against water, lyophilized and further separated by molecular sieve column chromatography on Sepharose CL-4B (Amersham Pharmacia Biotech AB, Uppsala, Sweden) in the presence of 4 M GuHCl (Research Plus Laboratories, Denville, N.J.), 0.1 M Na₂SO₄ (Sigma), 0.01 M EDTA (Sigma), and 0.05 M sodium acetate (Sigma), pH 5.8. Fractions from the CL-4B column were tested for the presence of SZP by dot blot analysis. Fractions positive for SZP were pooled, dialyzed against water and lyophilized. The purified product was analyzed by SDS-PAGE and Western blot analysis and revealed a single band with an apparent molecular weight of 345,000 Dalton.

### Example 14

Western Blot Analysis of Culture Medium from Human Synovium

Synovial tissue was obtained from human knees and placed into DMEM as stated above for cartilage slices until all tissue was collected. The tissue was finely minced into <1 mm³ pieces and placed in a spinner flask with DMEM/ITS as stated above for cartilage slices. Culture medium was harvested and the synovial tissue was refed three times per week. EDTA was added to the harvested culture media to a final concentration of 0.005 M and the media were frozen at −20° C. until further processed. Cultures were maintained for two weeks.

Aliquots of 150 μl of the crude media from two cultures were precipitated with 5 volumes of cold acetone and pelleted by centrifugation. The pellets were dissolved in SDS-PAGE sample buffer and analyzed by SDS-PAGE and Western blotting using the monoclonal antibody to SZP, described above, as a probe. Two samples of media from two donors revealed different reactivity to the antibody. Culture medium of synovium from one donor showed only the large molecular weight form of SZP (approx. 345,000 Da). The second sample of medium of synovium from a second donor showed only lower molecular weight fragments reacting with the antibody. Analysis of thirty different synovial fluid samples from organ donors or patients with degenerative joint disease, however, revealed that most samples contained primarily the 345 kDa form with some having minor bands at 280 kDa and 220 kDa.

### Example 15

Western Blot Analysis of Culture Medium from Chondrocytes

Human SZP was detected in the culture medium from the articular cartilage slices. The epitopes recognized by the GW4.23 anti-human SZP antibody were altered when samples were either boiled or reduced. Therefore all samples for Western blotting were treated with non-reducing Laemmli sample buffer and were not boiled. Samples were separated on 5% or 4-20% gradient gels and transferred to nitrocellulose. The blots were blocked with milk, treated with the GW4.23 antibody and then with a goat-anti-mouse IgG-horseradish peroxidase conjugate. Reactive bands were visualized on x-ray film after exposure to a Pierce Pico West chemiluminescent substrate.

SZP was purified from culture medium of superficial zone articular cartilage slices or from human synovial fluid. The purification involved a three step process as described above; however, the first step in the process was changed to ion exchange chromatography due to the larger volumes of culture medium to be processed. The second step was separation on a CsCl₂ density gradient, followed by gel filtration. Western blots showed SZP from cartilage slices and purified from synovial fluid. Both bands migrated above the 220 kDa protein standard. This preparation was separated by SDS-PAGE on a 5% gel and stained with several different stains including Coomassie blue, Stainsall and also a silver stain. Microsequencing of these bands yielded 5 sequences with homology to the precursor for megakaryocyte stimulating factor, and sequences of no other proteins were detected.

### Example 16

Immortalization of Human Chondrocytes from Different Zones

Thin slices from the superficial, middle and deep zones of the talar dome from human ankles were manually dissected. Slices from the middle zone were discarded and only the slices from the superficial and deep zones were processed for chondrocyte isolation. Chondrocytes from the superficial and deep zone were isolated separately by sequential enzymatic digestion with 0.02% pronase (Calbiochem, LaJolla, Calif.) and 0.25% collagenase P (Boehringer Mannheim, GmBh, Mannheim, Germany) in DMEM with 5% FBS by previously described methods (Aydelotte M B and Kuettner K E (1988); Aydelotte et al. (1998)). Chondrocytes were plated at high and low density in medium consisting of DMEM supplemented with 5% FBS and allowed to attach overnight at 37° C.

The following morning the culture medium was replaced with culture medium containing recombinant baculovirus constructed according to the methods of Condreay et al. (1999), which is incorporated herein by reference in its entirety for the methods of construction. Briefly, the virus was constructed according to the method for recombinant baculovirus generation described by Luckow et al. (1993). To construct the recombinant baculovirus DNA, the shuttle plasmid pFastBacMam-SV40 T Ag was transformed into the E. coli host DH10Bac (Life Technologies) and colonies that contain recombinant viral genomes (termed bacmids) are grown and the viral DNA is extracted. Recombinant baculovirus DNA is transfected into the insect cell line Sf9 and culture supernatants (which contain virus) are collected three days later. This virus stock is amplified by further propagation in Sf9 cells.

The chondrocytes were cultured in virus-containing medium for one hour at 37° C. The virus-containing medium was removed, and the chondrocytes were incubated for a second time in virus-containing medium for another hour under the same conditions. After the second incubation time the medium containing the virus was removed and replaced with medium consisting of DMEM supplemented with 5% FBS, and the cultures were incubated overnight at 37° C. in a humidified atmosphere of 5% CO₂/air. The cultures were refed the next day and throughout the remainder of the culture period with DMEM/5% FBS supplemented with 500 μg/ml G418 (Geneticin) (Gibco). Identical (but separate) cultures were transfected with the enhanced green fluorescent protein using the same methods described above. Phenomena observed in the cultures transfected with the enhanced green fluorescent protein were assumed to be also occurring in the cultures transfected with Tag.

Chondrocytes from the deep zone that were plated at high density showed signs of transformation with the virus the day after the virus was added, i.e. cultures were intensely fluorescent due to the expression of enhanced green fluorescent protein. Chondrocytes from the superficial zone seeded at high density showed only very faint green fluorescence. Approximately 4-5 days after the cultures were refed with medium containing G 418, cells began to die. Within two weeks after the addition of G418 to the cultures, only foci of cells remained in the cultures of deep zone chondrocytes and no chondrocytes from the superficial zone remained. The foci of cells from the deep zone proliferated in the presence of G 418 and were subcultured with trypsin EDTA. The chondrocytes from the deep zone immortalized with TAg and grown in the presence of G 418 continue to proliferate. Chondrocytes from the superficial zone seeded at low density showed signs of transformation the day following addition of the virus. Following the protocol outlined above, chondrocytes from the superficial zone seeded at low density formed foci of cells in the presence of G 418.

### Example 17

Lubrication by SZP

An apparatus was built similar to that described by Swann et al. (1981). This machine tests the ability of a protein solution to reduce frictional force between a weighted rabbit phalangeal bone and a rotating glass plate. The machine is essentially a modified record player (BIC). A 12 inch diameter, 0.25 inch thick plate glass record with a ⅜ inch central hole was manufactured by a local glass shop (Pollock Glass, Westmont, Ill.). The motor for the original record player was disconnected and the plate was driven instead by a belt drive attached to a digital stirring motor (Servodyne, Scientific Products) whose speed could be adjusted continuously from 1-180 rpm. The original phonograph needle was removed. Instead a brass pipe fitting held an adult rabbit phalangeal bone with its articular cartilage surface facing the glass record. The bone was held in place with a set screw and positioned at a radius of 3 inches from the center of the glass record. A small brass rod was positioned perpendicular to the tone arm and transmitted the forces on the rabbit bone to a force transducer (0-5 grams) (Transducer Technologies, Temechula, Calif.). A combination power supply and digital display meter (PAC110, Transducer Technologies) was connected to the force transducer and also to a chart recorder (Linear Instruments, Reno, Nev.; Model 220) to provide a continuous, real-time record of all the forces experienced during each experiment. The rabbit bones used in the experiment were dissected from the distal phalangeal bone of the middle toe of adult rabbits. Before the experiment the bones or intact rabbit feet were stored frozen and or in physiological saline. For each experiment about 1 ml of test solution was placed in a ring on the glass plate at the same radius as the rabbit bone stylus. Force measurements were recorded while the glass plate was rotating at 180, 160, 140, 120, 100, 80, 60, 40, and 20 rpm. These rotations correspond to speeds of 1 to 20 cm/sec. Measurements were taken for 2 minutes at each speed and the speed was always decreased from high to low in the above order. Then the process was repeated for a duplicate measurement for each sample. The first and last samples to be tested were always physiological saline as a control for the integrity of the cartilage tissue. Samples for testing included dilutions of synovial fluid, serum, bovine serum albumin, hyaluronic acid and purified SZP.

In this test device lubricating activity was dependent on three variables, the frictional force experienced by the rabbit cartilage, the speed of glass plate rotation and the dilution of the synovial fluid tested. Lubricating activity is increased when the frictional force on the rabbit cartilage is decreased, when lower speeds are necessary to register a change from the baseline frictional force and if lower concentrations (dilutions) of the synovial fluid are necessary to attain changes in frictional force above baseline.

High concentrations of synovial fluid lubricated so well in this test device that it was difficult to distinguish between the small changes in frictional force exhibited by the two lowest dilutions (1:2 compared to 1:10) tested in this experiment. It was not until a 1:20 dilution that much of an intermediate force (between 2-4 grams) was detected in this system. Five grams was the maximum force measured by the force transducer and data points were plotted at this value for a given speed when the maximum force is exceeded. To achieve an accurate measurement for the lubrication capacity of the synovial fluid, it was diluted to the point of generating an intermediate frictional value, optimally at the half maximum force of about 2.5 grams. To normalize the data for each bone used in the test, the frictional forces experienced with synovial fluid dilutions were compared as a ratio to the saline (PBS) control. An experimental lubrication capacity was defined as the Δ force/Δ rpm for the saline control divided by the Δ force/Δ rpm for the synovial fluid dilution necessary to give a force near the 2.5 g value. This ratio was then multiplied by the dilution factor necessary to achieve these intermediate force measurements. For example:

saline (2.5 g−1 g)/(180 −100 rpm)=1.5 g/80 rpm=0.018

diluted synovial fluid (2.1−0.75 g)/180−60 rpm)=1.35 g/120 rpm=0.011

Lubrication capacity=(saline control/synovial fluid dilution)×dilution factor=(0.018/0.011)×20=32.7

Thus a lubrication capacity was assigned for each synovial fluid sample, and it reflected the ability of synovial fluid to lubricate the cartilage in this test device. By testing a series of synovial fluid dilutions, a lubrication capacity was calculated for each synovial fluid sample. A lubrication capacity of 1 meant the sample had the same lubrication capacity as PBS. A value of 20 meant the synovial fluid must be diluted by a factor of 20 to reduce the lubricating activity to the level of the PBS control. The lubrication capacity was a ratio of the synovial fluid value to the PBS value and therefore does not have units. Since the measurements for the synovial fluid sample and PBS are made using the same rabbit articular cartilage, they are independent of the surface area of the cartilage. This allows the comparison of the many different synovial fluid samples tested on different rabbit cartilages.

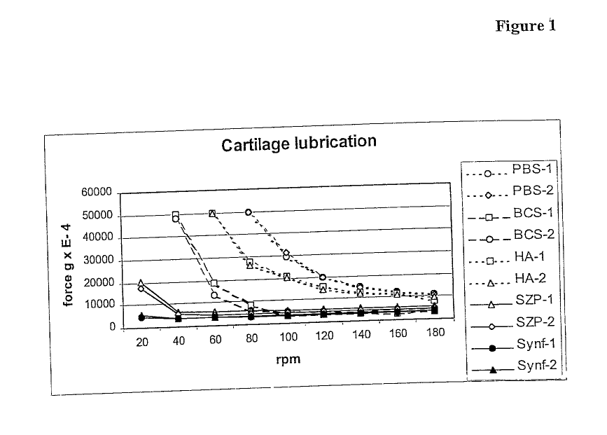

Physiological saline (PBS), which served as a negative control, had minimal lubricating activity and displayed the greatest frictional force at each speed tested compared to other samples. The best lubricating agent was undiluted synovial fluid. This showed a minimal and constant low frictional force at most of the middle and high speeds. Only at the low speeds did the frictional force increase. Although hyaluronic acid (HA) is sometimes described in the literature as a lubricant, in our assay, HA does not lubricate and behaves more like PBS. Low concentrations of BSA (1 mg/ml) did not lubricate. Surprisingly, undiluted serum did show high levels of lubricating activity. Purified preparations of SZP also displayed lubricating activity. See FIG. 1.

### Example 18

Measurement of SZP in Human Synovial Fluid

A. Sandwich ELISA for Human SZP

An ELISA assay was developed to measure the concentration of SZP in synovial fluids. Anti-human SZP monoclonal antibody was purified from the culture medium of hybridoma cultures, as described above.

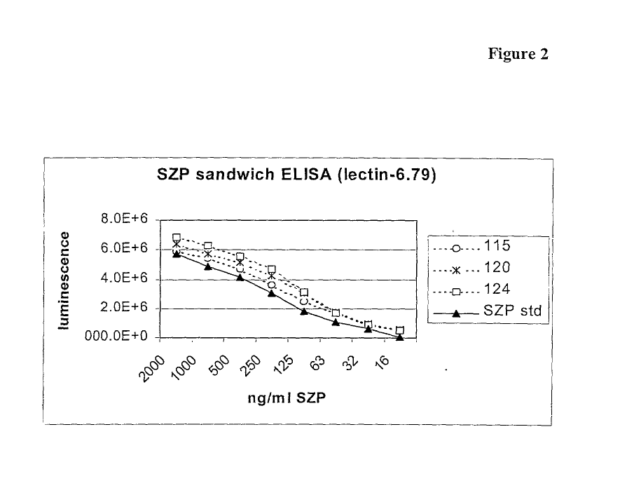

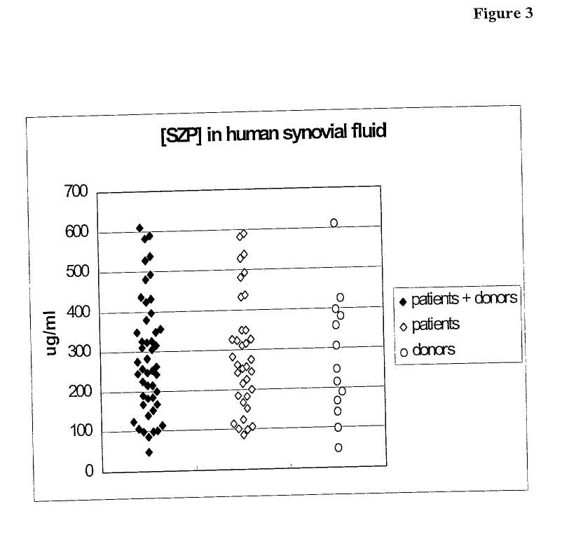

A peanut lectin (Sigma Chemical Co., St. Louis, Mo.) was used to coat black 96-well plates at a concentration of 1 μg/ml in 0.1 M NaHCO₃, pH 8.5. Plates were blocked with 1% BSA. Dilutions of synovial fluid or an SZP standard were made and incubated with the lectin-coated plates for 2 h. After washing the plates with PBS-Tween (0.05%), the plates were incubated with an anti-SZP monoclonal antibody for 1 h, washed and incubated with a goat-anti-mouse-HRP conjugate (Pierce Chemical Co., Rockford, Ill.). Bound HRP enzyme activity was detected with a chemiluminescent substrate (Pierce Chemical Co, Rockford, Ill.) and a luminometer. Two-fold serial dilutions (1:60 to 1:4000) were sufficient to measure the concentrations of SZP in synovial fluid. The assay was able to measure an SZP concentration in the range of 25-5000 ng/ml. See FIG. 2. Fifty samples of human synovial fluids from organ donors or patients with degenerative joint disease contained a range of SZP concentration from about 60-600 μg/ml. The mean value was 286 μg/ml with a standard deviation of 146 μg/ml. The data are shown in FIG. 3.

B. Western Blot

The concentration of SZP in human synovial fluid was also assessed using Western blotting of two-fold dilutions of synovial fluid compared to a purified SZP standard. Equivalent amounts of SZP were separated by SDS-PAGE and transferred to nitrocellulose, then serial dilutions of synovial fluid were compared to purified SZP to determine the relative detection limits for both preparations. Such an experiment showed synovial fluid to have about ten times the amount of SZP as the purified SZP stock solution with a concentration of 20 μg/ml. This experiment also estimated the concentration of SZP in synovial fluid to be about 200 μg/ml.

### Example 19

Localization of SZP at the Surface of Articular Cartilage using Immunohistochemistry and Electron Microscopy

To further demonstrate that SZP is indeed present at the surface of articular cartilage, immunohistochemical studies were performed using a gold-conjugated second antibody and visualizing the location of SZP by electron microscopy according to methods known in the art. The articular surface of bovine metacarpophalangeal cartilage stained with the antibody to bovine SZP and a gold conjugated second antibody. Gold particles decorated the surface boundary of the cartilage, at the synovial fluid interface. Collagen fibrils were evident slightly deeper in the tissue.

### Example 20

SZP Antibodies Reduce the Lubricating Effects of Synovial Fluid

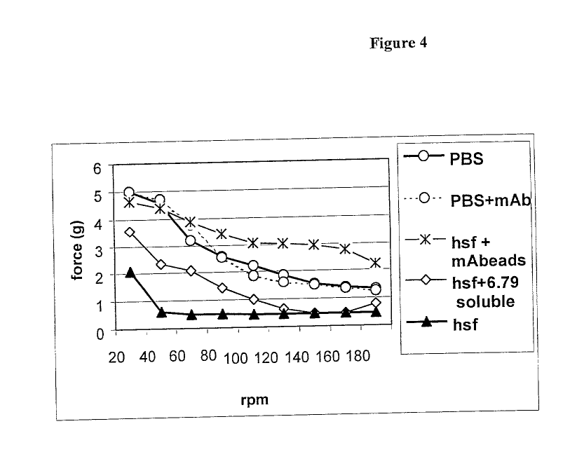

A 1:10 dilution of human synovial fluid was incubated with SZP monoclonal antibody, which was conjugated to Sepharose beads. The synovial fluid and conjugated antibody were incubated for 2 h, and the unbound solution was tested for lubrication using the techniques described above. The solid phase conjugated antibody removed almost all the lubricating activity from the synovial fluid. See FIG. 4. Soluble antibody had a similar effect on lubricating activity. An equal concentration of antibody, either conjugated or soluble, did not affect the lubrication activity of the PBS control. Thus, SZP plays a significant role in the lubricating activity of human synovial fluid.

### Example 21

SZP Binding Studies

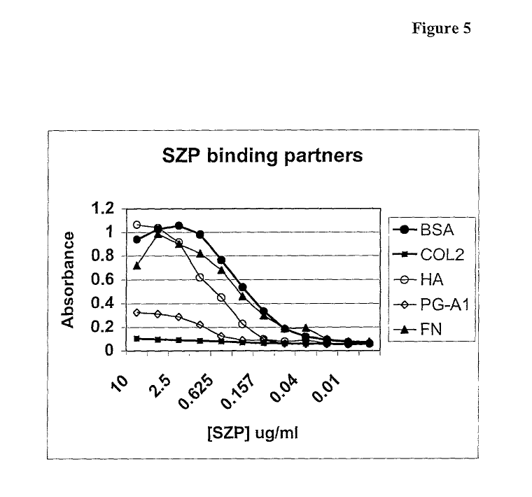

The ability of SZP to function as a lubricant is dependent on its ability to coat surfaces like the surface of cartilage, and this function is dependent on the formation of complexes with other macromolecules. Several purified matrix macromolecules known to be present in cartilage were coated on 96-well plates (50 μg/ml in pH 9.5 coating buffer). The molecules included bovine serum albumin (BSA, Sigma), hyaluronan (HA, Sigma), fibronectin (FN, gift from Dr. Gene Homandberg), pepsinized bovine collagen II (COL2), and aggrecan-link protein-hyaluronan complex (RCS-A1 fraction) isolated from the rat chondrosarcoma tumor and purified on an associative CsCl₂ gradient. The plates were blocked with PBS-Tween and incubated with different concentrations of human SZP. Any bound SZP was detected with an SZP monoclonal antibody, followed by a goat-anti-mouse IgG-HRP conjugate. After washing, the HRP activity was detected as an increase in color with an o-phenylenediamine substrate. The results are shown in FIG. 5. SZP bound in a concentration-dependent and saturable manner to bovine serum albumin, hyaluronan, and fibronectin. There was diminished but detectable binding to the aggrecan-link protein-HA complex. Some of the binding of this complex may be through unoccupied positions on the HA. The SZP did not bind to pepsinized collagen type II.

### Example 22

SZP Reduction in Cell Attachment

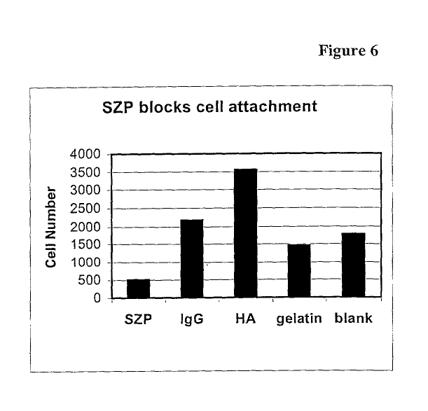

Tissue culture plates were coated with SZP and several other macromolecules to test if they would promote or inhibit chondrocyte attachment. Flacon tissue culture plates were coated overnight with 10 μg/ml of SZP, IgG, HA, gelatin, or no coating in a pH 9.5 coating buffer. The plates were washed with PBS-Tween and then an equal number of bovine chondrocytes in a single-cell suspension were added to each plate. They were incubated at 37° C. for 4 hours on a rocking platform in DME plus insulin, transferrin and selenium. The plates were washed with PBS and fixed in 10% formalin. The cells in 5 fields were counted using a 10× objective. The results are shown in FIG. 6. The SZP-coated plate had the smallest number of cells compared to other coated or non-coated (blank) plates. SZP diminished the ability of the cells to bind to the tissue culture plastic. These data show that SZP functions at the cartilage surface to prevent the attachment of cells to this tissue.

### Example 23

Characterization of SZP in Osteoarthritic Cartilage

These cloning experiments were designed to differentiate between Megakaryocyte Stimulating Factor (MSF) and SZP found in cartilage tissue. The amplified cDNAs were ligated into a baculovirus expression vector and recombinant proteins were then utilized for functional and antibody studies. SZP was amplified from pooled OA knee cartilage tissue samples by reverse transcriptase/polymerase chain reaction assays (sense primer 5′-atg gca tgg aaa aca ctt ccc att-3′ (SEQ ID NO: 7) and anti-sense primer 5′-cta agg aca gtt gta cca gac ttt-3′ (SEQ ID NO: 8)). Total RNA was isolated following the outlined methods in Analytical Biochemistry 202, 89-95 (1992), which is incorporated herein in its entirety. Complimentary DNA synthesis was done by incubating at 42° C. for 10 minutes for extension of oligo dT primers followed by reverse transcriptase at 42° C. for 60 minutes. PCR was initiated by Advantage HF polymerase denaturation of the RNA/cDNA complex at 95° C. for 3 min followed by 40 thermal cycles of 95° C., 15 sec, 60° C., 15 sec, and 72° C. 4 min. RT-PCR products were excised from Reliant (FMC) 1% TBE agarose gels, purified from agarose (Qiagen Gel Extraction kit), then inserted into pCR2. 1 (Invitrogen TA Topo kit). Template sequencing was performed on a PE Applied Biosystems 3700 DNA Sequencer using M13 forward and reverse primers. Positive clones #24 and #25 were excised from pCR2.1 by the restriction endonuclease NotI and subcloned into the NotI site of pFastBac-1 (BRL). A high titre virus was then generated by conventional means in Sf9 cells. An MOI of 5 was used to infect Tni cells for protein expression studies.

SZP products excised and sequenced revealed the absence of exon 2, exon 4, exon 5, and combinations of these deletions. The amino end contains contiguous sequences ending in . . . FCAE (SEQ ID NO: 9), splicing out 93 amino acids, then encoding exon 6 amino acids VKDNKKNR . . . (SEQ ID NO: 10).

Amino Acid Sequence encoded by 5′ Cartilage SZP
cDNA

(SEQ ID NO:2)

MAWKTLPIYLLLLLSVFVIQQVSSQDLSSCAGRCGEGYSRDATCNCDYNC
QHYMECCPDFKRVCTAELSCKGRCFESFERGRECDCDAQCKKYDKCCPDY
ESFCAEVKDNKKNRTKKKPTPKPPVVDEAGSGLDNGDFKVTTPDTSTTQH
NKVSTSPKITTAKPINPRPQSSPNSDTSKETSLTVNKE
5′ Nucleic Acid Sequence of Cartilage SZP cDNA

(SEQ ID NO:5)

5′-ATGGCATGGAAAACACTTCCCATTTACCTGTTGTTGCTGCTGTCTGT
TTTCGTGATTCAGCAAGTTTCATCTCAAGATTTATCAAGCTGTGCAGGGA
GATGTGGGGAAGGGTATTCTAGAGATGCCACCTGCAACTGTGATTATAAC
TGTCAACACTACATGGAGTGCTGCCCTGATTTCAAGAGAGTCTGCACTGC
GGAGCTTTCCTGTAAAGGCCGCTGCTTTGAGTCCTTCGAGAGAGGGAGGG
AGTGTGACTGCGACGCCCAATGTAAGAAGTATGACAAGTGCTGTCCCGAT
TATGAGAGTTTCTGTGCAGAAGTAAAAGATAACAAGAAGAACAGAACTAA
AAAGAAACCTACCCCCAAACCACCAGTTGTAGATGAAGCTGGAAGTGGAT
TGGACAATGGTGACTTCAAGGTCACAACTCCTGACACGTCTACCACCCAA
CACAATAAAGTCAGCACATCTCCCAAGATCACAACAGCAAAACCAATAAA
TCCCAGACCCCAGTCTTCACCTAATTCTGATACATCTAAAGAGACGTCTT
TGACAGTGAATAAAGAG-3′

The 3′ sequence is homologous with MSF. Calculating mucin-like repeat domains in this SZP cDNA means there are 30-32 less repeats (KEPAPTTT/P (SEQ ID NO: 11)) in the central region of the protein. The 5′ sequence contains the consensus sequences for N-glycosylation (NXS/T (SEQ ID NO: 4)) and chondroitin sulfate substitution (DEAGSG (SEQ ID NO: 1)). The junction between the 3′ sequence and the mucin region is a novel junction, not present in MSF.

Amino Acid Sequence Encoded by 3′ Cartilage SZP
cDNA

(SEQ ID NO:3)

PTTIHKSPDESTPELSAEPTPKALENSPKEPGVPTTKTPAATKPEMTTTA
KDKTTERDLRTTPETTTAAPKMTKETATTTEKTTESKITATTTQVTSTTT
QDTTPFKITTLKTTLAPKVTTTKKTITTTEIMNKPEETAKPKDRATNSKA
TTPKPQKPTKAPKKPTSTKKPKTMPRVRKPKTTPTPRKMTSTMPELNPTS
RIAEAMLQTTTRPNQTPNSKLVEVNPKSEDAGGAEGETPHMLLRPHVFMP
EVTPDMDYLPRVPNQGIIINPMLSDETNICNGKPVDGLTTLRNGTLVAFR
GHYFWMLSPFSPPSPARRITEVWGIPSPIDTVFTRCNCEGKTFFFKDSQY
WRLFTNDIKDAGYPKPIFKGFGGLTGQIVAALSTAKYKNWPESVYFFKRG
GSIQQYIYKQEPVQKCPGRRPALNYPVYGEMTQVRRRRFERAIGPSQTHT
IRIQYSPARLAYQDKGVLHNEVKVSILWRGLPNVVTSAISLPNIRKPDGY
DYYAFSKDQYYNIDVPSRTARAITTRSGQTLSKVWYNCP
3′ Nucleic Acid Sequence of Cartilage SZP cDNA

(SEQ ID NO:6)

5′-CCTACCACTATCCACAAAAGCCCTGATGAATCAACTCCTGAGCTTTC
TGCAGAACCCACACCAAAAGCTCTTGAAAACAGTCCCAAGGAACCTGGTG
TACCTACAACTAAGACTCCTGCAGCGACTAAACCTGAAATGACTACAACA
GCTAAAGACAAGACAACAGAAAGAGACTTACGTACTACACCTGAAACTAC
AACTGCTGCACCTAAGATGACAAAAGAGACAGCAACTACAACAGAAAAAA
CTACCGAATCCAAAATAACAGCTACAACCACACAAGTAACATCTACCACA
ACTCAAGATACCACACCATTCAAAATTACTACTCTTAAAACAACTACTCT
TGCACCCAAAGTAACTACAACAAAAAAGACAATTACTACCACTGAGATTA
TGAACAAACCTGAAGAAACAGCTAAACCAAAAGACAGAGCTACTAATTCT
AAAGCGACAACTCCTAAACCTCAAAAGCCAACCAAAGCACCCAAAAAACC
CACTTCTACCAAAAAGCCAAAAACAATGCCTAGAGTGAGAAAACCAAAGA
CGACACCAACTCCCCGCAAGATGACATCAACAATGCCAGAATTGAACCCT
ACCTCAAGAATAGCAGAAGCCATGCTCCAAACCACCACCAGACCTAACCA
AACTCCAAACTCCAAACTAGTTGAAGTAAATCCAAAGAGTGAAGATGCAG
GTGGTGCTGAAGGAGAAACACCTCATATGCTTCTCAGGCCCCATGTGTTC
ATGCCTGAAGTTACTCCCGACATGGATTACTTACCGAGAGTACCCAATCA
AGGCATTATCATCAATCCCATGCTTTCCGATGAGACCAATATATGCAATG
GTAAGCCAGTAGATGGACTGACTACTTTGCGCAATGGGACATTAGTTGCA
TTCCGAGGTCATTATTTCTGGATGCTAAGTCCATTCAGTCCACCATCTCC
AGCTCGCAGAATTACTGAAGTTTGGGGTATTCCTTCCCCCATTGATACTG
TTTTTACTAGGTGCAACTGTGAAGGAAAAACTTTCTTCTTTAAGGATTCT
CAGTACTGGCGTTTTACCAATGATATAAAAGATGCAGGGTACCCCAAACC
AATTTTCAAAGGATTTGGAGGACTAACTGGACAAATAGTGGCAGCGCTTT
CAACAGCTAAATATAAGAACTGGCCTGAATCTGTGTATTTTTTCAAGAGA
GGTGGCAGCATTCAGCAGTATATTTATAAACAGGAACCTGTACAGAAGTG
CCCTGGAAGAAGGCCTGCTCTAAATTATCCAGTGTATGGAGAAATGACAC
AGGTTAGGAGACGTCGCTTTGAACGTGCTATAGGACCTTCTCAAACACAC
ACCATCAGAATTCAATATTCACCTGCCAGACTGGCTTATCAAGACAAAGG
TGTCCTTCATAATGAAGTTAAAGTGAGTATACTGTGGAGAGGACTTCCAA
ATGTGGTTACCTCAGCTATATCACTGCCCAACATCAGAAAACCTGACGGC
TATGATTACTATGCCTTTTCTAAAGATCAATACTATAACATTGATGTGCC
TAGTAGAACAGCAAGAGCAATTACTACTCGTTCTGGGCAGACCTTATCCA
AAGTCTGGTACAACTGTCCTTAG-3′

### Example 24

SZP mRNA Expression in Transformed Human Chondrocyte Cell Line

Primers which span the SZP exons 1-6 and another set which amplify exons 6-12 were used to identify the splicing variants of SZP derived from human cartilage mRNA and to see if RT-PCR was able to detect higher levels of SZP mRNA in enriched populations of superficial zone chondrocytes compared to deep zone chondrocytes. Enriched populations of superficial and deep zone chondrocytes were obtained by manual dissection, released from their matrix by pronase-collagenase and cultured for 4 weeks in alginate beads. mRNA was isolated by Trizol (Life Technologies, Gaithersburg, Md.) extraction. mRNA from superficial (S) and deep (D) chondrocytes were amplified by RT-PCR with two primer sets, as described in Jay et al., 2001). The data show one product from the exon 6-12 primer and more of this product in superficial cells than in the enriched deep cells. Four splicing variants would be expected according to Jay et al, 2001, from exon 1-6 primer set 350, 475, 625, 750 and 825 bp. Very little product if any was detected in the deep cells with the exon 1-6 primer set. The diminished level of SZP mRNA in deep chondrocytes was consistent with protein extraction and immunohistochemical observations. The data suggest loss of SZP in the deeper zones of cartilage is due to low mRNA levels and is consistent with transcriptional regulation of SZP expression.

RT-PCR, with the SZP primers described above, was used to screen mRNA from cartilage tissue, primary chondrocyte cultures, as well as chondrogenic and non-chondrogenic cell lines for SZP expression. Some of the cartilage tissues and cell lines were positive for SZP expression, while other cell lines were negative. OA hip cartilage and macroscopically normal (Collin's grade 0) ankle cartilage were pulverized in liquid nitrogen and the mRNA isolated with Trizol. Both tissues showed the presence of variably spliced bands generated with the SZP exon 1-6 primer set. A HeLa cell line also was positive for SZP and the two lowest splice variants products at 350 and 475 bp were prominent. Human articular chondrocytes were transfected with a vector containing SV40 large T antigen to generate a cell line called HACTag. This cell line also expresses SZP. Two other cell lines (105 AJ and 105M) were derived from a human chondrosarcoma tumor by Dr. Joel Block were tested for SZP expression. These cell lines were negative for SZP mRNA when tested using the SZP exon 1-6 or the exon 6-12 primer sets. However the SZP-negative cell lines did show strong expression of GAPDH.

### Example 25

Generation of Recombinant SZP using a Baculovirus Vector in Sf9 Cells

Viruses were generated using the shuttle vector derived from pFastBac1 (Gibco BRL). Amplified products from normal articular cartilage encoding an open reading frame of 3.2 kb were cloned into the NotI site of pFastBac1. The recombinant baculovirus derived from pFastBac-SZP3.2 #25 was generated in Sf9 cells using standard methods (Summers and Smith, 1987) according to the Bac-to Bac instruction protocol. The high titre Re-BV stocks were prepared by infecting 150×106 Sf9 cells for 4 days. Baculovirus titres were measured in Sf9 cells by plaque assay using standard techniques (Summers and Smith, 1987). For expression, a Trichoplusia ni-derived cell line, adapted to suspension culture, was grown in EX-CELL 405 medium and infected in the same manner as Sf9 cells using an MOI of 2. The infected cultures were collected at 72 h post-infection and the cells were pelleted by centrifugation. The clarified medium was collected and the cell pellets washed once with PBS. These samples were then flash frozen for analysis in Westerns or functional assays. Western blots of clone #25 medium preparations showed strong reactivity of an anti-human SZP monoclonal antibody cocktail (mixture of S6.79, S17.109, S13.52, S13.230, GW4.23) with bands at 110 and 220 kDa. This data shows these methods are capable of generating high molecular weight forms of SZP that retain their native epitopes recognized by several anti-human SZP monoclonal antibodies.

### Example 26

Generation of pCEP4 Mammalian Expression Plasmid

A fragment of SZP3.2 #25 was generated using the NotI restriction endonuclease. This fragment was inserted into the NotI site of pCEP4 (Invitrogen, Carlsbad Calif.) for transient and stable protein production in 293 EBNA cells. Standard cell transfections were carried out using FuGENE 6 (Roche, Indianapolis Ind.). Following transfection, cells were passed in 100 mm dishes with 300 μg ml-1 hygromycin for 11 days. After the selection period, cells were plated at 1×106 cells onto 100 mm dishes and media samples were taken at 3 days. See Parham et al., 1998.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### References

1. Aigner T, Vornehm S I, Zeiler G, Dudhia J, von der Mark K, Bayliss M T. Suppression of cartilage matrix gene expression in upper zone chondrocytes of osteoarthritic cartilage. Arthritis Rheum 40:562-569, 1997.

2. Aydelotte M B and Kuettner K E (1988) Differences between sub-populations of cultured bovine articular chondrocytes. I. Morphology and cartilage matrix production. Connect Tissue Res. 18:205-222.

3. Aydelotte M B et al. (1988) Differences between sub-populations of cultured bovine articular chondrocytes. II. Proteoglycan metabolism. Connect Tissue Res 18:223-234.

4. Condreay J. P., et al. Transient and stable gene expression in mammalian cells transduced with a recombinant baculovirus vector. PNAS 96: 127-132 (1999).

5. de Belder A N, Wik K O. Preparation and properties of fluorescein-labelled hyaluronate. Carbohydr. Res. 44(2):251-57 (1975).

6. Eisenbraun M D, et al. Examination of parameters affecting the elicitation of humoral immune response by particle bombardment mediated genetic immunization. DNA Cell Biol. 1993; 12:791-797.

7. Fägerstam L G et al (1992) Biospecific interaction analysis using surface plasmon resonance detection applied to kinetic, binding site and concentration analysis. Chromatography 1992; 597:397-410.

8. Flannery, C R, Hughes, C E, Schumacher, B L, Tudor, D., Aydelotte, M B, Kuettner, K E, and Caterson, B. Articular cartilage superficial zone protein (SZP) is homologous to megakaryocyte stimulating factor precursor and is a multifunctional proteoglycan with potential growth-promoting, cytoprotective, and lubricating properties in cartilage metabolism. Biochem. Biophys. Res. Commun. 254:535-541, 1999.

9. Freemont A J, Hampson V, Tilman R, Goupille P, Taiwo Y, Hoyland J A. Gene expression of matrix metalloproteinases 1, 3, and 9 by chondrocytes in osteoarthritic human knee articular cartilage is zone and grade specific. Ann Rheum Dis 56:542-549, 1997.

10. Guilak F, Ratcliffe A, Lane N, Rosenwasser M P, Mow V C. Mechanical and biochemical changes in the superficial zone of articular cartilage in canine experimental osteoarthritis. J Orthop Res 12:474-484, 1994.

11. Harlow and Lane “Antibodies, A Laboratory Manual” Cold Spring Harbor Publications, New York (1988).

12. Hauselmann H J, Flechtenmacher J, Michal L, Thonar E J, Shinmei M, Kuettner K E, Aydelotte M B. The superficial layer of human articular cartilage is more susceptible to interleukin-1-induced damage than the deeper layers. Arthritis Rheum 39:478-488, 1996.

13. Hollander A P, Pidoux I, Reiner A, Rorabeck C, Bourne R, Poole A R. Damage to type II collagen in aging and osteoarthritis starts at the articular surface, originates around chondrocytes, and extends into the cartilage with progressive degeneration. J Clin Invest 96:2859-2869, 1995.

14. Jay, G D, Britt, D E, and Cha, C -J Lubricin is a product of megakaryocyte stimulating factor gene expression by human synovial fibroblasts. J. Rheumatol. 27:594-600, 2000.

15. Jay G D, Tantravahi U, Britt, D E, Barrach H J, and Cha C -J. Homology of lubricin and superficial zone protein (SZP): products of megakaryocyte stimulating factor (MSF) gene expression by human synovial fibroblasts and articular chondrocytes localized to chromosome 1q25. J Orthop Res 19:677-687, 2001.

16. Kaplan J B, et al. (1997) Characterization of a soluble vascular endothelial growth factor receptor-immunoglobulin chimera. Growth Factors 14:243-256.

17. Khaw B A et al. (1980) Myocardial infarct imaging of antibodies to canine cardiac myosin with indium-111-diethylenetriamine pentaacetic acid. Science 209:295-97.

18. Kilpatrick K E et al. (1997) Rapid development of affinity matured monoclonal antibodies using RIMMS. Hybridoma 16:381-89.

19. Kilpatrick K E, et al. (1998) Gene gun delivered DNA-based immunizations mediate rapid production of murine monoclonal antibodies to the Flt-3 receptor. Hybridoma 17:569-576.

20. Krejcarek G E et al. (1977) Covalent attachment of chelating groups to macromolecules. Biochem. Biophys. Res. Commun. 77:581-85.

21. Lark M W, Bayne E K, Flanagan J, Harper C F, Hoerrner L A, Hutchinson N L, Singer I I, Donatelli S A, Weidner J R, Williams H R, Mumford R A, Lohmander L S. Aggrecan degradation in human cartilage. Evidence for both matrix metalloproteinase and aggrecanase activity in normal, osteoarthritic, and rhematoid joints. J Clin Invest 100:93-106, 1997.

22. Lindley et al. (2000) Production of monoclonal antibodies using recombinant baculovirus displaying gp64-fusion proteins. J. Immunological Methods 2000;234:123-135.

23. Lorenzo P, Bayliss M T, Heinegard D. A novel cartilage protein (CILP) present in the mid-zone of human articular cartilage increases with age. J Biol Chem. (1998) 273:23463-68.

24. Luckow et al., Efficient generation of infectious recombinant baculoviruses by site-specific transposon-mediated insertion of foreign genes into a baculovirus genome propagated in Escherichia coli, J. Virol. 67: 4566-4579 (1993).

25. Marcelino, J, Carpten, J D, Suwairi, W M, Gutierrez, O M, Schwartz, S, Robbins, C, Sood, R, Makalowska, I, Baxevanis, A, Johnstone, B, Laxer, R M, Zemel, L, Kin, C A, Herd, J K, Ihle, J, Williams, C, Johnson, M, Raman, V, Alonso, L G, Brunoni, D, Gerstein, A, Papadopoulos, N, Bahabri, S A, Trent, J M, Warman, M L. CACP, endoding a secreted proteoglycan, is mutated in camptodactyly-arthropathy-coxavara-pericarditis syndrome. Nature Genetics 23:319-322, 1999.

26. March, S C, Parikh, I, and Cuatrecasas, P. A simplified method for cyanogen bromide activation of agarose for affinity chromatography. Anal. Biochem. 60:149-152, 1974.

27. Merberg, D M, Fitz, L J, Temple, P, Giannotti, J, Murtha, P, Fitzgerald, M, Scaltreto, H, Kelleher, K, Preissner, K, Kriz, R, Jacobs, K, and Turner, K. A comparison of vitronectin and megakaryocyte stimulating factor. In Preissner, K T, Rosenblatt, S, Kost, C, Wegerhoff, J, Mosher, D F, eds. Biology of vitronectins and their recptors. New York, Elsevier Science, pp. 45-52, 1993.

28. Ohta S, Imai K, Yamashita K, Matsumoto T, Azumano I, Okada Y. Expression of matrix metalloproteinase 7 (matrilysin) in human osteoarthritic cartilage. Lab Invest 78:79-87, 1998.

29. Panula H E, Hyttinen M M, Arokoski J P, Langsjo T K, Peltarri A, Kiviranta I, Helminen H J. Articular cartilage superficial zone collagen birefringence reduced and cartilage thickness increased before surface fibrillation in experimental osteoarthritis. Ann Rheum Dis 57:237-245, 1998.

30. Parham J H, Iannone M A, Overton L K, and Hutchins J T (1998) Optimization of Transient Gene Expression in Mammalian Cells and Potential for Scale-up Using Flow Electroporatin. Cytotechnology 28:147-155.

31. Pertmer T M, et al. Influenza virus nucleo-protein-specific immunoglobulin G subclasses and cytokine response elicited by DNA vaccination are dependent on the route of vector DNA delivery. J. Virol 1996;70:6119-6125.

32. Sambrook, J., Fritsch, E. F., and Maniatis, T., (1989) “Molecular Cloning: A laboratory manual”. Cold Spring Harbor Lab. Press, Plainview, N.Y.

33. Schmid T M et al. (1994) Immunohistochemical distribution of a novel proteoglycan in the surface lamina of articular cartilage. Proceedings of the Orthopedic Research Society, p. 97-17.

34. Schumacher, B L, Block, J A Schmid, T M, Aydelotte, M B, and Kuettner, K E. A novel proteoglycan synthesized and secreted by chondrocyte of the superficial zone of articular cartilage. Arch. Biochem. Biophys. 311:144-152, 1994.

35. Schumacher, B L, Hughes, C E, Kuettner, K E, Caterson, B and Aydelotte, M B. Immunodetection and partial cDNA sequence of the proteoglycan, superficial zone protein, synthesized by cells lining the synovial joints. J. Orthop. Res. 17:110-120, 1999.

36. Su J -L et al. (1999) Use of a PPAR gamma-specific monoclonal antibody to demonstrate thiazolidinediones induce PPAR gamma receptor expression in vitro. Hybridoma 18:273-280.

37. Su J -L, Becherer D, Edwards C, Burkhart W, McGeehan G M and Champion B R (1995) Monoclonal antibodies against human collagenase and stromelysin. Hybridoma 14:383-390. Summers, M D and Smith G A, (1987) A Manual of Methods for Baculovirus Vectors and Insect Cell Culture Procedures. Tex. Agric. Exp. Stn. Bull. B1555.

38. Swann, D A, Hendren, R B, Radin, E L, Sotman, S L, and Duda, E A. The lubricating activity of synovial fluid glycoproteins. Arthritis and Rheum. 24:22-30, 1981.

39. Towle C A, Hung H H, Bonassar L J, Treadwell B V, Mangham D C. Detection of interleukin-1 in the cartilage of patients with osteoarthritis: a possible autocrine/paracrine role in pathogenesis. Osteoarthritis Cartilage 5:293-300, 1997.

What is claimed is:

1. An immortalized chondrocyte derived from a chondrocyte isolated from a specific zone of articular cartilage.

2. The immortalized chondrocyte of claim 1, wherein the chondrocyte retains one or more characteristics of non-immortalized chondrocytes.

3. The immortalized chondrocyte of claim 1, wherein the chondrocyte is derived from superficial zone cartilage.

4. The immortalized chondrocyte of claim 1, wherein the chondrocyte expresses SZP, or fragments or derivatives of SZP having lubricating properties.

5. The immortalized chondrocyte of claim 1, wherein the chondrocyte is non-tumor derived.

6. The immortalized chondrocyte of claim 1, wherein the chondrocyte is immortalized by transduction with a virus.

7. The immortalized chondrocyte of claim 6, wherein the virus is a baculovirus.

8. A method of culturing chondrocytes in a serum-free medium, comprising culturing the chondrocytes in serum-free medium supplemented with insulin, transferrin, and selenium.

9. A method of making isolated SZP, or fragments or derivatives of SZP having lubricating properties, comprising the steps of:
(a) culturing chondrocytes in serum-free medium under conditions that allow expression of SZP or its fragments or derivatives;
(b) harvesting the medium from the cultured chondrocytes; and
(c) isolating SZP or its fragments or derivatives from the medium.

10. The method of claim 9, wherein the chondrocytes are immortalized.

11. A method of making SZP, or a fragment or derivative thereof having lubricating properties, comprising
(a) culturing a cell comprising an exogeneous nucleic acid that encodes the SZP or its fragment or derivative, wherein the exogeneous nucleic acid is operably linked to an expression control sequence, and wherein the culture conditions permit expression of SZP under the control of the expression control sequence;
(b) harvesting the medium from the cultured cells, and
(c) isolating the SZP from the cell or culture medium.

12. The method of claim 11, wherein the cell is an insect cell.

13. The method of claim 12, wherein the insect cell is a baculovirus-infected cell.

14. The method of claim 11, wherein the cell is a mammalian cell.

15. The method of claim 11, wherein the isolated SZP lacks glycosylation.

16. The method of claim 15, wherein the isolated SZP lacking glycosylation has a molecular weight of about 110 kDa.

17. The method of claim 11, wherein the isolated SZP is glycosylated.

18. The method of claim 17, wherein the isolated, glycosylated SZP has a molecular weight of greater than 280 kDa.

19. The method of claim 11, wherein the culture conditions include serum free culture medium.

20. A method of modulating lubrication of an articular surface of a joint, comprising contacting the articular surface of the joint with an SZP binding protein, under conditions which allow SZP to bind to the SZP binding protein.

21. The method of claim 20, wherein the SZP binding protein is selected from the group consisting of an SZP antibody, lectins, hyaluronan, fibronectin, and albumin.

22. A method of modulating lubrication of a biological surface, comprising contacting the surface with an SZP binding protein, under conditions which allow SZP to bind to the SZP binding protein.

23. A method of modulating lubrication of a biologically compatible surface comprising contacting the surface with an SZP binding protein, under conditions which allow SZP to bind to the SZP binding protein.

24. A method of inhibiting cell adhesion to a biological surface comprising contacting the biological surface with SZP, or a fragment or derivative of SZP having cell adhesion inhibiting properties, under conditions which allow SZP, or the fragment or derivative thereof, to inhibit cell adhesion to the surface.

25. The method of claim 24, wherein the biological surface is selected from the group consisting of cartilage, tendon, ligament, pericardium, and blood vessel.

26. The method of claim 24, wherein the SZP or its fragment or derivative lacks glycosylation.

27. The method of claim 26, wherein the SZP or its fragment or derivative lacking glycosylation has a molecular weight of about 110 kDa.

28. The method of claim 24, wherein the SZP or its fragment or derivative is glycosylated.

29. The method of claim 28, wherein the glycosylated SZP or its fragment or derivative has a molecular weight of greater than 280 kDa.

30. A method of inhibiting cell adhesion to a biologically compatible surface comprising contacting the biologically compatible surface with SZP, or a fragment or derivative of SZP having cell adhesion inhibiting properties, under conditions which allow the SZP, or fragment or derivative thereof, to inhibit cell adhesion to the surface.

31. The method of claim 30, wherein the biologically compatible surface is the surface of a prosthetic device.

32. The method of claim 30, wherein the SZP or its fragment or derivative lacks glycosylation.

33. The method of claim 32, wherein the SZP or its fragment or derivative lacking glycosylation has a molecular weight of about 110 kDa.

34. The method of claim 30, wherein the SZP or its fragment or derivative is glycosylated.

35. The method of claim 34, wherein the glycosylated SZP or its fragment or derivative has a molecular weight of greater than 280 kDa.

36. A method of inhibiting cell adhesion to an articular surface of a joint, comprising contacting the articular surface of the joint with SZP, or fragments or derivatives of SZP, having cell adhesion inhibiting properties, under conditions which allow the SZP, or fragment or derivative thereof, to inhibit cell adhesion to the articular surface.

37. The method of claim 36, wherein the contacting step is in vivo.

38. The method of claim 36, wherein the contacting step is extra-corporeal.

39. The method of claim 36, wherein the joint is a prosthetic joint.

40. The method of claim 36, wherein the joint is a natural joint.

41. The method of claim 40, wherein the joint shows one or more signs of a degenerative joint condition.

42. The method of claim 36, wherein the SZP or its fragment or derivative lacks glycosylation.

43. The method of claim 42, wherein the SZP or its fragment or derivative lacking glycosylation has a molecular weight of 110 kDa or less.

44. The method of claim 36, wherein the SZP or its fragment or derivative is glycosylated.

45. The method of claim 44, wherein the glycosylated SZP or its fragment or derivative has a molecular weight of greater than 280 kDa.

46. A method of promoting lubrication of a biological surface comprising contacting the biological surface with SZP or its fragment or derivative made according to the method of claim 11, under conditions which allow the SZP, or fragment or derivative thereof, to bind to the biological surface.

47. A method of promoting lubrication of a biologically compatible surface comprising contacting the biologically compatible surface with SZP or its fragment or derivative made according to the method of claim 11, under conditions which allow the SZP, or fragment or derivative thereof, to bind to the surface.

48. The method of claim 47, wherein the biologically compatible surface is the surface of a prosthetic device.

49. A method of promoting lubrication of an articular surface of a joint, comprising contacting the articular surface of the joint with SZP or its fragment or derivative made according to the method of claim 11, under conditions which allow the SZP, or fragment or derivative thereof, to bind to the articular surface.

50. The method of claim 49, wherein the contacting step is in vivo.

51. The method of claim 49, wherein the contacting step is extra-corporeal.

52. The method of claim 49, wherein the joint is a prosthetic joint.

53. The method of claim 49, wherein the joint is a natural joint.

54. The method of claim 53, wherein the joint shows one or more signs of a degenerative joint condition.

55. A method of treating a subject with a degenerative joint condition or of delaying symptoms of a degenerative joint condition in a subject, comprising administering to the subject a therapeutically effective amount of SZP or fragments or derivatives of SZP made according to the method of claim 11.

56. The method of claim 55, wherein the degenerative joint condition is an arthritic condition.

57. The method of claim 56, wherein the arthritic condition is osteoarthritis.

58. The method of claim 56, wherein the arthritic condition is rheumatoid arthritis.

59. A purified SZP polypeptide comprising the amino acid sequences of SEQ ID NOS: 2 and 3 and having a molecular weight of less than 110 kDa.

60. A purified SZP polypeptide comprising the amino acid sequences of SEQ ID NOS: 2 and 3 with one or more conservative amino acid substitutions.

61. A purified SZP polypeptide comprising an amino acid sequence having at least 80% identity to SEQ ID NOS: 2 and 3.

62. An isolated nucleic acid comprising a nucleotide sequence that encodes the SZP polypeptide of claim 59.

63. The isolated nucleic acid of claim 62, comprising the nucleotide sequences of SEQ ID NO: 5 and 6 or their degenerate variants.

64. An expression vector comprising the nucleic acid of claim 63 operably linked to an expression control sequence.

65. A cultured cell comprising the vector of claim 64.

66. The isolated nucleic acid of claim 63, further comprising a nucleotide sequence encoding one or more mucin repeats.

67. An isolated nucleic acid comprising a sequence that hybridizes under highly stringent conditions to a hybridization probe having the nucleotide sequence of SEQ ID NOS: 5 and 6 or their complements.

68. A method of imaging an articular surface or synovium of a joint, comprising:
(a) contacting the articular surface or synovium of the joint with detectably tagged SZP or its fragment or derivative, under conditions in which the detectably tagged SZP or its fragment or derivative binds to the articular surface or synovium;
(b) visualizing the detectable tag in a plurality of locations on the articular surface or synovium;
(c) the visualization of detectable tag showing the articular surface or synovium of the joint.

69. The method of claim 68, wherein the detectable tag is a radiolabel.

70. The method of claim 69, wherein the radiolabel is selected from the group consisting of gamma-emitters, beta-emitters, and alpha-emitters.

71. The method of claim 68, wherein the detectable tag is a fluorescent label.

72. The method of claim 68, wherein the detectable tag is a magnetic label.

73. The method of claim 68, wherein the visualization step comprises a means of visualization selected from the group consisting of nuclear magnetic resonance, X-radiography, positron emission tomography, computerized axial tomography, magnetic resonance imaging, and ultrasonography.

74. A method of imaging an articular surface or synovium of a joint, comprising:
(a) contacting the articular surface or synovium of the joint with detectably tagged SZP binding protein, under conditions in which the detectably tagged SZP binding protein binds to SZP on the articular surface or synovium;
(b) visualizing the detectable tag in a plurality of locations on the articular surface or synovium;
(c) the visualization of detectable tag showing the articular surface or synovium of the joint.

75. The method of claim 74, wherein the detectable tag is a radiolabel.

76. The method of claim 75, wherein the radiolabel is selected from the group consisting of gamma-emitters, beta-emitters, and alpha-emitters.

77. The method of claim 74, wherein the detectable tag is a fluorescent label.

78. The method of claim 74, wherein the detectable tag is a magnetic label.

79. The method of claim 74, wherein the visualization step comprises a means of visualization selected from the group consisting of nuclear magnetic resonance, X-radiography, positron emission tomography, computerized axial tomography, magnetic resonance imaging, and ultrasonography.

80. The method of claim 74, wherein the SZP binding protein is selected from the group consisting of lectins, hyaluronan, fibronectin, and albumin.
